(19) United States
(12) Patent Application Publication
(10) Pub. No.: US 2024/0341870 A1
(43) Pub. Date: Oct. 17, 2024

(54) SYSTEM AND METHOD FOR IMAGING AND REGISTRATION FOR NAVIGATION

(71) Applicant: Medtronic Navigation, Inc., Lafayette, CO (US)

(72) Inventors: Andre D.A. SOUZA, Boylston, MA (US); Patrick A. HELM, Canton, MA (US)

(21) Appl. No.: 18/630,348

(22) Filed: Apr. 9, 2024

Related U.S. Application Data
(60) Provisional application No. 63/459,306, filed on Apr. 14, 2023.
(60) Provisional application No. 63/459,308, filed on Apr. 14, 2023.
(60) Provisional application No. 63/459,318, filed on Apr. 14, 2023.
(60) Provisional application No. 63/459,354, filed on Apr. 14, 2023.

(51) Int. Cl. A61B 34/30; A61B 34/20; A61B 90/00; G16H 30/20; G16H 30/40

(52) U.S. Cl. CPC A61B 34/30; A61B 34/20; A61B 90/37; G16H 30/20; G16H 30/40; A61B 2034/2055; A61B 2090/374; A61B 2090/3762

(57) ABSTRACT

Disclosed is a system for assisting in guiding and performing a procedure on a subject. The subject may be any appropriate subject such as inanimate object and/or an animate object. An imaging system may be used to image the subject. A system may include various manipulable or movable members, such as robotic systems, and may be used to move and position the imaging system.

### CROSS-REFERENCE TO RELATED APPLICATIONS

This application claims the benefit of U.S. Provisional Application No. 63/459,306, filed Apr. 14, 2023; U.S. Provisional Application No. 63/459,308, filed Apr. 14, 2023; U.S. Provisional Application No. 63/459,318, filed Apr. 14, 2023; and U.S. Provisional Application No. 63/459,354, filed Apr. 14, 2023. The entire disclosures of the above applications are incorporated herein by reference.

This application includes subject matter similar to that disclosed in concurrently filed U.S. Patent Application No. __/___,___ (Attorney Docket No. A0008290US01/5074A-000247-US-PS1); U.S. Patent Application No. __/___,___ (Attorney Docket No. A0010638US02/5074A-000300-US); U.S. Patent Application No. __/___,___ (Attorney Docket No. A0010640US02/5074A-000301-US); and U.S. Patent Application No. __/___,___ (Attorney Docket No. A0010655US02/5074A-000307-US). The entire disclosures of the above applications are incorporated herein by reference.

### FIELD

The subject disclosure is related generally to a tracking and navigation system, and particularly to imaging and registering coordinate systems.

### BACKGROUND

This section provides background information related to the present disclosure which is not necessarily prior art.

An instrument can be navigated relative to a subject for performing various procedures. For example, the subject can include a patient on which a surgical procedure is being performed. During a surgical procedure, an instrument can be tracked in an object or subject space. In various embodiments, the subject space can be a patient space defined by a patient. The location of the instrument that is tracked can be displayed on a display device relative to an image of the patient.

The position of the patient can be determined with a tracking system. Generally, a patient is registered to the image, via tracking an instrument relative to the patient to generate a translation map between the subject or object space (e.g., patient space) and the image space. This often requires time during a surgical procedure for a user, such as a surgeon, to identify one or more points in the subject space and correlating, often identical points, in the image space.

After registration, the position of the instrument can be appropriately displayed on the display device while tracking the instrument. The position of the instrument relative to the subject can be displayed as a graphical representation, sometimes referred to as an icon on the display device.

### SUMMARY

This section provides a general summary of the disclosure, and is not a comprehensive disclosure of its full scope or all of its features.

According to various embodiments, an imaging system may be used to acquire image data of a subject. The imaging system may include an ultrasound imaging system that includes an ultrasound (US) probe that generally includes an ultrasound transducer to emit and receive ultrasound frequencies. It is understood, however, that the imaging system may include separate components that emit and receive ultrasound frequencies.

According to various embodiments, the US probe may be moved relative to a subject, such as a by a user and/or with a robotic system. The robotic system may include an appropriate robotic system, such as a Mazor X™ Robotic Guidance System, sold by Mazor Robotics Ltd. having a place of business in Israel and/or Medtronic, Inc. having a place of business in Minnesota, USA and/or as disclosed in U.S. Pat. No. 11,135,025, incorporated herein by reference. The US probe may be moved to achieve acquisition of selected image data.

According to various embodiments, the imaging system may be incorporated into various components. For example, the imaging system may be incorporated into a surgical drape. Again, the imaging system may include a US probe that may have a single housing for a transducer and/or may include separate components.

The imaging system, that may include the US probe, may also be used in various techniques of imaging or following selected portions in a procedure. For example, the US probe may be associated with the robotic system. The robotic system may move the US probe in a selected manner during the procedure. During the procedure the US probe may be moved based upon predetermined characteristics to follow or maintain a selected view of a selected portion during the procedure, such as a portion of a subject or a portion of an instrument.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is diagrammatic view illustrating an overview of a robotic system and a navigation system, according to various embodiments;

FIG. 2 is a detailed environmental view of a surgical drape with an ultrasound assembly and optional robotic system with instrument, according to various embodiments;

FIG. 3A is a detailed cross-sectional view of a surgical drape with an ultrasound assembly, according to various embodiments;

FIG. 3B is a detailed cross-sectional view of a surgical drape with an ultrasound assembly, according to various embodiments;

FIG. 3C is a detailed cross-sectional view of a surgical drape with an ultrasound assembly, according to various embodiments;

FIG. 4 is a detailed environmental view of a surgical drape with an ultrasound assembly, according to various embodiments;

FIG. 5 is a detailed environmental view of a surgical drape with an ultrasound assembly, according to various embodiments;

FIG. 6 is a detailed view of a subject support including an ultrasound transducer, according to various embodiments;

FIG. 7 is an environmental view of an operating room with a robotic system holding an ultrasound probe, according to various embodiments;

FIG. 8 is a flow chart of a process for moving and acquiring image data with a robotic system, according to various embodiments;

FIG. 9 is a detailed view of a robotic system holding an ultrasound probe in a first position, according to various embodiments;

FIG. 10 is a detailed environmental view of a robotic system holding an ultrasound probe in a second position;

FIG. 11A and FIG. 11B is a flow chart of a process to acquire image data of a selected portion of the subject, according to various embodiments;

FIG. 12 is a detailed view of an operating room with a robotic system holding an ultrasound probe and a robotic system holding an instrument in a first position, according to various embodiments;

FIG. 13 is a detailed view of an operating room with a robotic system holding an ultrasound probe and a robotic system holding an instrument in a second position, according to various embodiments;

FIG. 14 is a detailed view of an operating room with a robotic system holding an ultrasound probe and a robotic system holding an instrument in a third position, according to various embodiments;

FIG. 15A and FIG. 15 B is a flow chart of a process to image a selected portion of an instrument, according to various embodiments;

FIG. 16 is a representative slice of a magnetic resonance (MR) image, according to various embodiments;

FIG. 17 is ultrasound image (sonogram), according to various embodiments;

FIG. 18A is a representative slice of a mock computed tomography (CT) image, according to various embodiments;

FIG. 18B is a detail view of a representative slice of the mock computed tomography (CT) image of FIG. 18A;

FIG. 19 is a representative slice of a segmented mock computed tomography (CT) image, according to various embodiments;

FIG. 20 is a segmented model based on selected image data, according to various embodiments; and

FIG. 21 is a flowchart of a process to acquire image data and register image data, according to various embodiments.

Corresponding reference numerals indicate corresponding parts throughout the several views of the drawings.

### DETAILED DESCRIPTION

Example embodiments will now be described more fully with reference to the accompanying drawings.

The subject disclosure is directed to an exemplary embodiment of a surgical procedure on a subject, such as a human patient. It is understood, however, that the system and methods described herein are merely exemplary and not intended to limit the scope of the claims included herein. In various embodiments, it is understood, that the systems and methods may be incorporated into and/or used on non-animate objects. The systems may be used to, for example, to register coordinate systems between two systems for use on manufacturing systems, maintenance systems, and the like. For example, automotive assembly may use one or more robotic systems including individual coordinate systems that may be registered together for coordinated or consorted actions. Accordingly, the exemplary illustration of a surgical procedure herein is not intended to limit the scope of the appended claims.

Discussed herein, according to various embodiments, are processes and systems for allowing registration between various coordinate systems. In various embodiments, a first coordinate system may be registered to a second coordinate system, such as a robotic coordinate system to an image coordinate system or space. A navigation space or coordinate system may then be registered to the robotic or first coordinate system and, therefore, be registered to the image coordinate system without being separately or independently registered to the image space. Similarly, the navigation space or coordinate system may be registered to the image coordinate system or space directly or independently. The robotic or first coordinate system may then be registered to the navigation space and, therefore, be registered to the image coordinate system or space without being separately or independently registered to the image space.

In various embodiments, the different systems used relative to the subject may include different coordinate systems (e.g., locating systems). For example, a robotic system may be moved relative to a subject that includes a robotic coordinate system. The robot may be fixed, including removably fixed, at a position relative to the subject. Thus, movement of a portion of the robot relative to the base of the robot (i.e., the fixed portion of the robot) may be known due to various features of the robot. For example, encoders (e.g., optical encoders, potentiometer encoders, or the like) may be used to determine movement or amount of movement of various joints (e.g., pivots) of a robot. A position of an end effector (e.g., a terminal end) of the robot may be known relative to the base of the robot. Given a known position of the subject relative to the base and the known position of the base relative to the subject, the position of the end effector relative to the subject may be known during movement of a robot and/or during a stationary period of the end effector. Thus, the robot may define a coordinate system relative to the subject.

Various other portions may also be tracked relative to the subject. For example, a tracking system may be incorporated into a navigation system that includes one or more instruments that may be tracked relative to the subject. The navigation system may include one or more tracking systems that track various portions, such as tracking devices, associated with instruments. The tracking system may include a localizer that is configured to determine the position of the tracking device in a navigation system coordinate system. Determination of the navigation system coordinate system may include those described at various references including U.S. Pat. Nos. 8,737,708; 9,737,235; 8,503,745; and 8,175,681; all incorporated herein by reference. In particular, a localizer may be able to track an object within a volume relative to the subject. The navigation volume, in which a device, may be tracked may include or be referred to as the navigation coordinate system or navigation space. A determination or correlation between the two coordinate systems may allow for or also be referred to as a registration between two coordinate systems.

In various embodiments, the first coordinate system, which may be a robotic coordinate system, may be registered to a second coordinate system, which may be a navigation coordinate system. Accordingly, coordinates in one coordinate system may then be transformed to a different or second coordinate system due to a registration. Registration may allow for the use of two coordinate systems and/or the switching between two coordinate systems. For example, during a procedure, a first coordinate system may be used for a first portion or a selected portion of a procedure and a second coordinate system may be used during a second portion of a procedure. Further, two coordinate systems may be used to perform or track a single portion of a procedure, such as for verification and/or collection of additional information.

Furthermore, images may be acquired of selected portions of a subject. The images may be displayed for viewing by a user, such as a surgeon. The images may have superimposed on a portion of the image a graphical representation of a tracked portion or member, such as an instrument. According to various embodiments, the graphical representation may be superimposed on the image at an appropriate position due to registration of an image space (also referred to as an image coordinate system) to a subject space. A method to register a subject space defined by a subject to an image space may include those disclosed in U.S. Pat. Nos. U.S. Pat. Nos. 8,737,708; 9,737,235; 8,503,745; and 8,175,681; all incorporated herein by reference.

During a selected procedure, the first coordinate system may be registered to the subject space or subject coordinate system due to a selected procedure, such as imaging of the subject. In various embodiments, the first coordinate system may be registered to the subject by imaging the subject with a fiducial portion that is fixed relative to the first member or system, such as the robotic system. The known position of the fiducial relative to the robotic system may be used to register the subject space relative to the robotic system due to the image of the subject including the fiducial portion. Thus, the position of the robotic system or a portion thereof, such as the end effector, may be known or determined relative to the subject. Due to registration of a second coordinate system to the robotic coordinate system may allow for tracking of additional elements not fixed to the robot relative to a position determined or tracked by the robot.

The tracking of an instrument during a procedure, such as a surgical or operative procedure, allows for navigation of a procedure. When image data is used to define an image space it can be correlated or registered to a physical space defined by a subject, such as a patient. According to various embodiments, therefore, the patient defines a patient space in which an instrument can be tracked and navigated. The image space defined by the image data can be registered to the patient space defined by the patient. The registration can occur with the use of fiducials that can be identified in the image data and in the patient space.

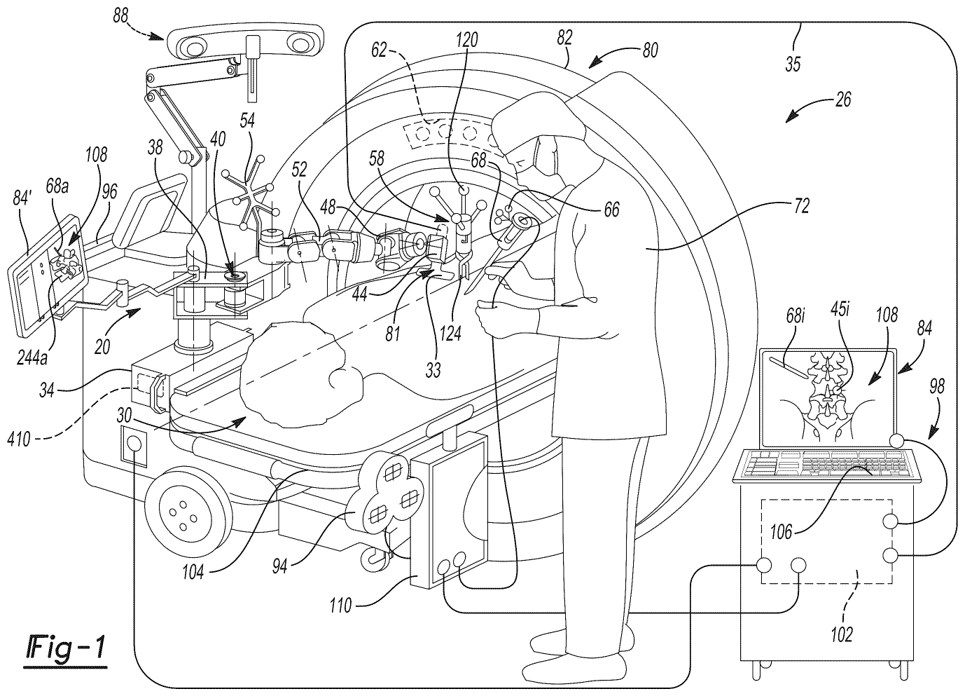

FIG. 1 is a diagrammatic view illustrating an overview of a procedure room or arena. In various embodiments, the procedure room may include a surgical suite in which may be placed a robotic system 20 and a navigation system 26 that can be used for various procedures. The robotic system 20 may include a Mazor X™ robotic guidance system, sold by Medtronic, Inc. The robotic system 20 may be used to assist in guiding a selected instrument, such as drills, screws, etc. relative to a subject 30. In addition of alternatively, the robotic system 20 may hold and/or move an imaging system, such as an ultrasound (US) probe 33. The robotic system 20 may include a mount 34 that fixes a portion, such as a robotic base 38, relative to the subject 30. The robotic system 20 may include one or more arms 40 that are moveable or pivotable relative to the subject 30, such as including an end effector 44. The end effector may be any appropriate portion, such as a tube, guide, or passage member. Affixed to and/or in place of the end effector may be the imaging system that may be the US probe 33. The end effector 44 may be moved relative to the base 38 with one or more motors. The position of the end effector 44 may be known or determined relative to the base 38 with one or more encoders at one or more joints, such as a wrist joint 48 and/or an elbow joint 52 of the robotic system 20.

The navigation system 26 can be used to track the location of one or more tracking devices, tracking devices may include a robot tracking device 54, a subject tracking device 58, an imaging system tracking device 62, and/or an tool tracking device 66. A tool or moveable member 68 may be any appropriate tool such as a drill, forceps, or other tool operated by a user 72. The tool 68 may also include an implant, such as a spinal implant or orthopedic implant. It should further be noted that the navigation system 26 may be used to navigate any type of instrument, implant, or delivery system, including: guide wires, arthroscopic systems, orthopedic implants, spinal implants, deep brain stimulation (DBS) probes, etc. Moreover, the instruments may be used to navigate or map any region of the body. The navigation system 26 and the various instruments may be used in any appropriate procedure, such as one that is generally minimally invasive or an open procedure.

An additional or alternative, imaging system 80 may be used to acquire pre-, intra-, or post-operative or real-time image data of a subject, such as the subject 30. It will be understood, however, that any appropriate subject can be imaged and any appropriate procedure may be performed relative to the subject. In the example shown, the imaging device 80 comprises an O-arm® imaging device sold by Medtronic Navigation, Inc. having a place of business in Louisville, Colorado, USA. The imaging device 80 may have a generally annular gantry housing 82 in which an image capturing portion is moveably placed. The imaging device 80 can include those disclosed in U.S. Pat. Nos. 7,188,998; 7,108,421; 7,106,825; 7,001,045; and 6,940,941; all of which are incorporated herein by reference, or any appropriate portions thereof. It is further appreciated that the imaging device 80 may include in addition or alternatively a fluoroscopic C-arm. Other exemplary imaging devices may include fluoroscopes such as bi-plane fluoroscopic systems, ceiling mounted fluoroscopic systems, cath-lab fluoroscopic systems, fixed C-arm fluoroscopic systems, isocentric C-arm fluoroscopic systems, 3D fluoroscopic systems, etc. Other appropriate imaging devices can also include MRI, CT, ultrasound, etc.

The position of the imaging system 33, 80, and/or portions therein such as the image capturing portion, can be precisely known relative to any other portion of the imaging device 33, 80. The imaging device 33, 80, according to various embodiments, can know and/or recall precise coordinates relative to a fixed or selected coordinate system. For example, the robotic system 20 may know or determine its position and positioned the SU probe 33 as a selected pose. Similarly, the imaging system 80 may also position the imaging portions at a selected pose. This can allow the imaging system 80 to know its position relative to the patient 30 or other references. In addition, as discussed herein, the precise knowledge of the position of the image capturing portion can be used in conjunction with a tracking system to determine the position of the image capturing portion and the image data relative to the tracked subject, such as the patient 30.

Herein, reference to the imaging system 33 may refer to any appropriate imaging system, unless stated otherwise. Thus, the US probe 33 as the imaging system is merely exemplary regarding the subject disclosure. As one skilled in the art will understanding, generally the US probe 33 may emit a US wave in a plane and receive an echo relative to any portions engaged by the wave. The received echo at the US probe 33 or other appropriate received may be used to generate image data and may be used to generate an US image also referred to as a sonogram.

The imaging device 80 can be tracked with a tracking device 62. Also, a tracking device 81 can be associated directly with the US probe 33. The US probe 33 may, therefore, be directly tracked with a navigation system as discussed herein. In addition or alternatively, the US probe 33 may be positioned and traced with the robotic system 20. Regardless, image data defining an image space acquired of the patient 30 can, according to various embodiments, be registered (e.g., manually, inherently, or automatically) relative to an object space. The object space can be the space defined by a patient 30 in the navigation system 26.

The patient 30 can also be tracked as the patient moves with a patient tracking device, DRF, or tracker 58. Alternatively, or in addition thereto, the patient 30 may be fixed within navigation space defined by the navigation system 26 to allow for registration. As discussed further herein, registration of the image space to the patient space or subject space allows for navigation of the instrument 68 with the image data. When navigating the instrument 68, a position of the instrument 68 can be illustrated relative to image data acquired of the patient 30 on a display device 84. An additional and/or alternative display device 84′ may also be present to display an image. Various tracking systems, such as one including an optical localizer 88 or an electromagnetic (EM) localizer 92 can be used to track the instrument 68.

More than one tracking system can be used to track the instrument 68 in the navigation system 26. According to various embodiments, these can include an electromagnetic tracking (EM) system having the EM localizer 94 and/or an optical tracking system having the optical localizer 88. Either or both of the tracking systems can be used to track selected tracking devices, as discussed herein. It will be understood, unless discussed otherwise, that a tracking device can be a portion trackable with a selected tracking system. A tracking device need not refer to the entire member or structure to which the tracking device is affixed or associated.

The position of the patient 30 relative to the imaging device 33 can be determined by the navigation system 26. The position of the imaging system 33 may be determined, as discussed herein. The patient 30 can be tracked with the dynamic reference frame 58, as discussed further herein. Accordingly, the position of the patient 30 relative to the imaging device 33 can be determined.

Image data acquired from the imaging system 33, or any appropriate imaging system, can be acquired at and/or forwarded from an image device controller 96, that may include a processor module, to the navigation computer and/or processor system 102 that can be a part of a controller or work station 98 having the display 84 and a user interface 106. It will also be understood that the image data is not necessarily first retained in the controller 96, but may also be directly transmitted to the work station 98. The work station 98 can provide facilities for displaying the image data as an image 108 on the display 84, saving, digitally manipulating, or printing a hard copy image of the received image data. The user interface 106, which may be a keyboard, mouse, touch pen, touch screen or other suitable device, allows the user 72 to provide inputs to control the imaging device 80, via the image device controller 96, or adjust the display settings of the display 84. The work station 98 may also direct the image device controller 96 to adjust the image capturing portion of the imaging device 80 to obtain various two-dimensional images along different planes in order to generate representative two-dimensional and three-dimensional image data.

With continuing reference to FIG. 1, the navigation system 26 can further include the tracking system including either or both of the electromagnetic (EM) localizer 94 and/or the optical localizer 88. The tracking systems may include a controller and interface portion 110. The controller 110 can be connected to the processor portion 102, which can include a processor included within a computer. The EM tracking system may include the STEALTHSTATION® AXIEM™ Navigation System, sold by Medtronic Navigation, Inc. having a place of business in Louisville, Colorado; or can be the EM tracking system described in U.S. patent application Ser. No. 10/941,782, filed Sep. 15, 2004, and entitled “METHOD AND APPARATUS FOR SURGICAL NAVIGATION”; U.S. Pat. No. 5,913,820, entitled “Position Location System,” issued Jun. 22, 1999; and U.S. Pat. No. 5,592,939, entitled “Method and System for Navigating a Catheter Probe,” issued Jan. 14, 1997; all of which are herein incorporated by reference. It will be understood that the navigation system 26 may also be or include any appropriate tracking system, including a STEALTHSTATION® TREON® or S7™ tracking systems having an optical localizer, that may be used as the optical localizer 88, and sold by Medtronic Navigation, Inc. of Louisville, Colorado. Other tracking systems include an acoustic, radiation, radar, etc. The tracking systems can be used according to generally known or described techniques in the above incorporated references. Details will not be included herein except when to clarify selected operation of the subject disclosure.

Wired or physical connections can interconnect the tracking systems, imaging device 80, etc. Alternatively, various portions, such as the instrument 68 may employ a wireless communications channel, such as that disclosed in U.S. Pat. No. 6,474,341, entitled “Surgical Communication Power System,” issued Nov. 5, 2002, herein incorporated by reference, as opposed to being coupled directly to the controller 110. Also, the tracking devices 62, 66, 54 can generate a field and/or signal that is sensed by the localizer(s) 88, 94.

Various portions of the navigation system 26, such as the instrument 68, and others as will be described in detail below, can be equipped with at least one, and generally multiple, of the tracking devices 66. The instrument can also include more than one type or modality of tracking device 66, such as an EM tracking device and/or an optical tracking device. The instrument 68 can include a graspable or manipulable portion at a proximal end and the tracking devices may be fixed near the manipulable portion of the instrument 68.

Additional representative or alternative localization and tracking system is set forth in U.S. Pat. No. 5,983,126, entitled “Catheter Location System and Method,” issued Nov. 9, 1999, which is hereby incorporated by reference. The navigation system 26 may be a hybrid system that includes components from various tracking systems.

According to various embodiments, the navigation system 26 can be used to track the instrument 68 relative to the patient 30. The instrument 68 can be tracked with the tracking system, as discussed above. Image data of the patient 30, or an appropriate subject, can be used to assist the user 72 in guiding the instrument 68. The image data, however, is registered to the patient 30. The image data defines an image space that is registered to the patient space defined by the patient 30. The registration can be performed as discussed herein, automatically, manually, or combinations thereof.

Generally, registration allows a translation map to be generated of the physical location of the instrument 68 relative to the image space of the image data. The translation map allows the tracked position of the instrument 68 to be displayed on the display device 84 relative to the image data 108. A graphical representation 68i, also referred to as an icon, can be used to illustrate the location of the instrument 68 relative to the image data 108.

With continuing reference to FIG. 1, a subject registration system or method can use the tracking device 58. The tracking device 58 may include portions or members 120 that may be trackable, but may also act as or be operable as a fiducial assembly. The fiducial assembly 120 can include a clamp or other fixation portion 124 and the imageable fiducial body 120. It is understood, however, that the members 120 may be separate from the tracking device 58. The fixation portion 124 can be provided to fix any appropriate portion, such as a portion of the anatomy. As illustrated in FIG. 1, the fiducial assembly 120 can be interconnected with a portion of a spine 126 such as a spinous process 130. The fixation portion 124 can be interconnected with a spinous process 130 in any appropriate manner. For example, a pin or a screw can be driven into the spinous process 130.

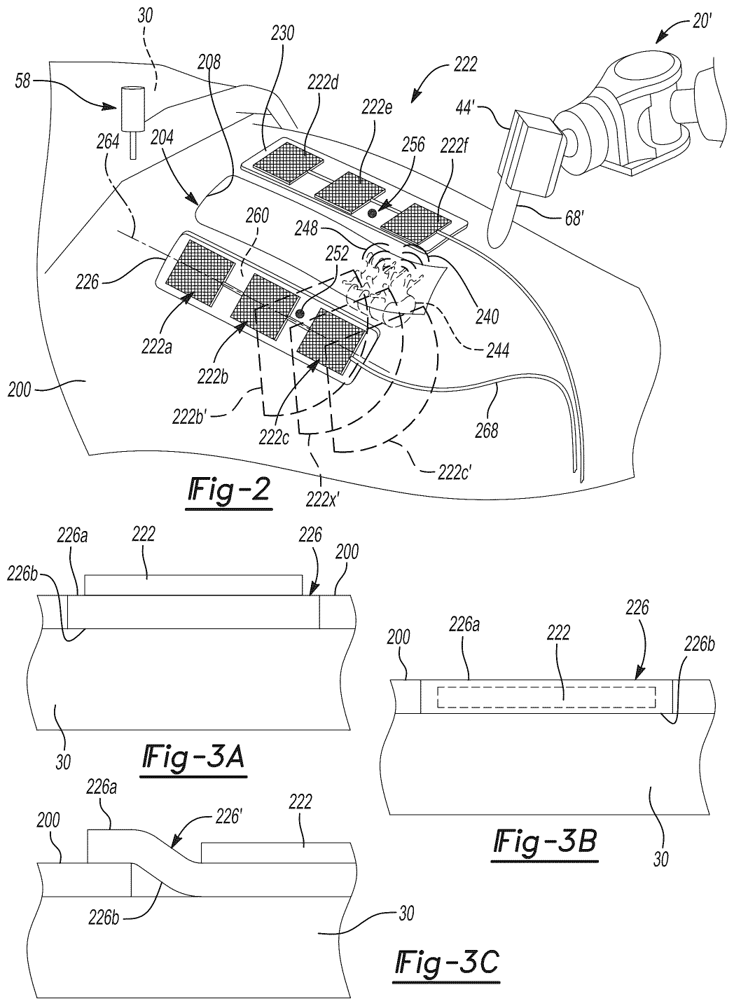

As illustrated in FIG. 1, the imaging device 33 may include the US probe 33 that may be positioned relative to the subject 30, such as by the robotic system 20. As discussed herein, therefore, the robotic system 20 may move the US probe 33 to a selected position relative to the subject 30. According to various embodiments, the imaging system may be positioned relative to the subject in any appropriate manner. For example, with reference to FIG. 2, a surgical drape 200 may be positioned on the subject 30. The surgical drape 200 may be a sterile sheet similar to known surgical drapes. The surgical drape 200 may include an opening or surgical access 204. The surgical access 204 may allow for the user 72 to move the instrument 68 relative to a portion of the subject 30, such as a spinal column including one or more vertebrae, through the surgical drape 200. The surgical access 204 may also be referred to as a port or portal and the drape 200 may be a sheet of material configured to be position on and/or over the subject 30. The surgical access 204 may define a boundary 208 through which the instrument 68 may pass.

At a selected location relative to the access 204 may be one or more ultrasound arrays 222 that may operate as the imaging system 33. The arrays 222 may be positioned or placed adjacent or otherwise near the access 204. As illustrated in the FIG. 2, the arrays 222 may be placed a selected distance from the edge 208, such as one millimeter (mm), 5 mm, 10 mm, 50 mm, or more than 50 mm. Generally, the arrays 222 are positioned to image a selected portion of the subject without obstructing access through the access 204 to the subject 30.

The ultrasound arrays 222 may include any appropriate number of arrays such as a first, second, and third array 222a, 222b, 222c on a first side of the opening 204. In addition or alternatively, ultrasound arrays 222d, 222e, and 222f may be positioned relative to a second portion of the opening 204. It is understood that more or less than the six ultrasound arrays 222 may be included relative to the subject 30 and six are shown merely as an example.

The ultrasound arrays may be arrayed in a group, such as three arrays 222a-222c positioned on a first acoustic window portion 226 and a second set of the arrays 222, including the arrays 222d-222f may be positioned on a second acoustic window 230. The acoustic windows 226, 230 may be formed into the surgical drape 200.

The acoustic windows 226, 230 may be formed as or of a substantially ultrasound coupling material portions that may be formed on or formed in an ultrasound opening (also referred to as a coupling port or portal) adjacent to the surgical opening 204. The sheet of the drape 200 may have formed therein a passage or opening (e.g., hole) for the windows 226, 230 that may be at least partially filled with a gel-like material, as discussed herein. Thus, the acoustic windows 226, 230 may substantially contact the subject 30. The windows 226, 230 may have appropriate acoustic coupling with of the subject 30. For example, the acoustic windows 226, 230 may be formed of a gel material that includes an appropriate ultrasound transmission property to achieve an appropriate imaging or wave coupling within the subject 30. Thus, the arrays 222 positioned on the acoustic windows 226, 230 being have appropriate acoustic coupling with the subject 30.

Turning in reference to FIGS. 3A, 3B, and 3C, the ultrasound arrays 222 may be associated with respective acoustic windows, such as the acoustic windows 226, 226′, and 230 in an appropriate manner. The acoustic windows may be associated with the subject 30 and/or the drape or other covering 200 to assist in providing an acoustic coupling of the array 222 with the subject 30. The acoustic window, therefore, may assist in ensuring an appropriate or active image acquisition clarity with the arrays 222 relative to the subject 30.

The acoustic windows, such as the acoustic windows 226, 230, 226′ may be formed of selected materials. For example, an adhesive formed with an acrylic material member, such as an acrylic member that has adhesives to contact the subject 30, the drape 200, and/or the array 222 may be provided. Similarly or alternatively, silicone materials and/or adhesives may also be used as the acoustic window. It is understood by one skilled in the art, however, that appropriate low acoustic impedance material may be provided as the acoustic windows. The low impedance acoustic impedance material may be provided as a tape (e.g., thin flexible member) including an adhesive on at least one surface or portion to adhere the tape or material to the subject 30 to ensure the maintaining of a selected pose of the acoustic window relative to the subject 30. The low acoustic impedance material may also be provided to have an adhesive on at least two surfaces to allow for adhesion to the subject 30 and adhesion to one or more of the arrays 222.

For example, with reference to FIG. 3A, the acoustic window 226 may include a first surface 226a and a second surface 226b. The first surface 226a may contact or adhere to the array 222. In various embodiments, an adhesive may be applied between the array 222 and the first surface 226a. The adhesive may be pre-applied or applied during a procedure. Similarly, or alternatively, an adhesive may be applied between the surface 226b and the subject 30. Again, the adhesive may be pre-applied or applied during a selected procedure.

In various embodiments, the acoustic window 226 may be included within the drape 200. For example, the acoustic window may be formed with and/or incorporated into the drape 200. The drape 200 may have a passage or opening that may be filled or connected with the acoustic window 226 in any appropriate manner such as with an adhesive, stitching, mechanical connection, or the like.

In various embodiments, as illustrated in FIG. 3B, the acoustic window 226 may have the two surfaces 226a and 226b to allow for contact with the subject 30. Again, the acoustic window 226 may be incorporated into the drape 200, as selected. The array 222, however, may be incorporated into the acoustic window 226. According to various embodiments, for example, the array 222 may be molded into or formed with the acoustic window 226. As discussed above, various acrylics or silicone materials or compositions including the same, may be used to mold or form the acoustic window 226 around and/or to incorporate to the array 222. Thus, the array 222 may be incorporated into the window 226.

The acoustic window may also be provided as a tape or adhesive such as the acoustic window 226′ as illustrated in FIG. 3C. The acoustic window 226′ may include the two surfaces 226a and 226b. As discussed above, both of the surfaces and/or at least one of the surfaces may be included as an adhesive. Therefore, the acoustic window 226′ may operate or act as a tape or adhering member to hold the drape 200 relative to the subject 30. For example, during a procedure, the drape 200 may be positioned relative to the subject and an opening or edge may be covered with the acoustic window 226′ to connect or hold the drape 200 relative to the subject 30. The array 222 may also be positioned relative to the acoustic window 226′, such as by contacting the first surface 226a. Again, both of the surfaces 226a and 226b may include pre-applied adhesives, adhesives applied during a procedure, or other selected connection mechanisms. The acoustic window 226′, according to any appropriate embodiment, may be provided as a tape that has a pre-applied adhesive to allow it to be adhered or connected with a selected portion, such as the drape 200, the subject 30, the array 222, or any appropriate portion. Regardless the acoustic window may have little acoustic impedance, according to various embodiments, to allow for an appropriate ultrasound transmission through the acoustic window to the subject 30 and for collecting ultrasound sonogram information due to a selected or appropriate acoustic coupling between the array 222 and the subject 30. According to various embodiments, the arrays 222 may be provided as transducer arrays. Therefore, each of the arrays may transmit an ultrasound frequency that propagates into the subject 30. As is generally understood by one skilled in the art, when the ultrasound wave contacts a solid material, such as a bone, the ultrasound wave may reflect off of the material. The reflected wave may then be received at a receiver portion of the transducer 222. Therefore, the ultrasound arrays 222 may operate as transmitters and receivers, also known as transducers. It is understood by one skilled in the art, however, that portions of the arrays 222 may operate separately and/or that various ones of the ultrasound arrays 222 operate as a transmitter and other of the arrays 222 operate as receivers.

As schematically illustrated in FIG. 2, ultrasound waves may be emitted by one or more of the ultrasound arrays 222 as a transmission ultrasound wave 240. The transmission wave 240 may engage a portion of the subject 30, such as a vertebrate 244. After encountering a certain object, the transmission wave 240 may then be reflected from the vertebrae 244 as a reflection wave 248. The reflection wave 248 may be received at one or more of the ultrasound arrays 222. For example, the transmission wave 240 may be emitted or transmitted from the portion 222f. They reflected wave 248 may be received at the ultrasound array 222f. Alternatively or additionally, the reflected wave 248 may be received at another one of the arrays 222. As is understood by one skilled in the art, each of the arrays 222 generally acquire image data in a plane. The plane may have a field of view relative to the portion of the array 222. Via the window, the field of view may include a portion of the subject.

As illustrated in FIG. 2, however, each of the arrays may be positioned relative to one another. Each of the arrays 222 may have known positions relative to one another, such as being provided with the ultrasound windows 226, 230 as a unit. The navigation system 26 and/or the imaging system 33, may know the position of the ultrasound arrays 222 relative to one another. In addition, the imaging system 33 may also have the known position of the ultrasound arrays 222 relative to another. Therefore, the transmission and receiving of the ultrasound waves may be known based upon the known position of the ultrasound arrays relative to one another.

According to various embodiments, the position and/or relative position of the arrays 222 may be established (e.g., predetermined and saved for recall) based on the type of procedure and/or the anatomical views needed or selected to carry out a selected procedure. For example, in spinal procedures the arrays may be placed so that details images of pedicles, or nerve roots may be visualized. In a further example, the arrays may be positioned in cardiac applications to optimize imaging one or more chambers of a heart.

The US transducers arrays 222 may also be associated with one or more tracking portions, such as a first tracking device 252 associated with the first ultrasound window assembly 226 and a second tracking device 256 associated with the second ultrasound window assembly 230. The tracking devices 250, 256 may be and/or include fiducial portions or members that are imaged with an imaging system, such as the imaging system 80 discussed above. The tracking devices 250, 256 may be tracking devices that may be tracked with the navigation system 26. The tracking devices 250, 256 may allow tracking of various portions, such as the ultrasound window assemblies 226, 230 to allow for a position of the ultrasound arrays 222, such as relative to the subject 30 (e.g., in subject or navigation space) to be determined with the navigation system. Therefore, image data acquired with the respective ultrasound arrays 222 may be registered to the subject 30 within the position information based on the navigation system 26 tracking the respective ultrasound arrays 222. The tracking devices 252, 256 may be tracked with the navigation system 26 the determine the pose of the ultrasound arrays 222 at an appropriate time, such as during a selected procedure.

One or more drape fiducials or tracking devices, similar to the devices 252, 256 may be incorporated in the drape 200 separate from the windows. The drape fiducials or tracking devices may allow the drape 200 and/or portions of the drape 200 to be tracked. The pose of the drape 200, including portions thereof such as the windows, may allow a moveable imaging system 33 to be moved relative thereto, such as automatically. As discussed herein, the US probe 400 may be moved with the robotic system 20. If the drape 200, or selected portion thereof has a known pose, including the opening 204, the US probe 400 may be moved automatically thereto for imaging.

Further, as is understood by one skilled in the art, the image data acquired with one or more of the ultrasound arrays 222 may be registered in navigation system such as disclosed in the U.S. Pat. No. 7,085,400 and U.S. U.S. Pat. No. 9,138,204, both incorporated herein by reference. The image data acquired within the respective ultrasound arrays 222 may be of the subject 30. As the ultrasound arrays 222 are registered to the subject 330 using the navigation system 26, the image data required of the subject 30, such as of the vertebrae 244, may also have its pose determined in navigation space within the navigation system 26.

The ultrasound arrays 222 may include one or more that are positioned relative to one another, such as on the ultrasound window assemblies, for example the ultrasound window 226. The ultrasound arrays 222a, 222b, and 222c may be at a set position relative to one another, such as in a linear position, a selected distance apart that may be a known distance 260 apart generally along or on an axis 264. The assembly may be connected to the imaging system 33 such as with a connection, including a wire connection 268. It is understood that a wireless connection may also or alternatively be provided. Therefore, the position of each of the arrays 222 may be known relative to one another and image data that is generated based upon a received signal by any of the arrays may be known relative to the others. Further, if the transmission wave 240 is sent by one of the arrays, such as the array 222a and received by another one of the arrays, such as the array 222b, the reflected wave 248 may still be known relative to the first ultrasound array and an appropriate image may be generated.

As discussed above, the ultrasound windows, such as the ultrasound window 226, may be incorporated into the drape 200. Therefore, the ultrasound window 226 may be in contact with the subject 30, as discussed above. The ultrasound window 226 may be incorporated into the drape 200 in any appropriate manner such as welding, adhesive, stitching, or the like. Further, the connections, such as the connection 268, may be a permanent or removable connection. Thus, the ultrasound arrays 222 may be used to acquire image data of selected portions of the subject 30 at an appropriate time. In various embodiments, for example, the ultrasound arrays 222 may substantially collect real time image data during a procedure. The ultrasound arrays 222 may be positioned relative to the surgical access opening 204 so that the user 72 may be able to view the subject 30 in substantially real time. Further, various instruments, such as the instrumental tool 68, may be echogenic. The instrument 68 may also be viewable in an image, such as the image 108, generated with the ultrasound image data. The navigation system 26 may allow for registration of the instrument 68 relative to the image 108 and an icon or graphical representation 68i may be displayed relative to the image 108. Additionally or alternatively, the image 108 may include image data of the instrument 68 such that the image 108 includes substantially real time image data of the instrument 68 to allow an instrument image 68a to be displayed with the image 108. That is as the instrument is imaged with the subject, an image of the instrument may be included in the image 108. The image 108 may include portions of the subject, such as the vertebrae 244 as a vertebrae image 244a and the instrument image 68a.

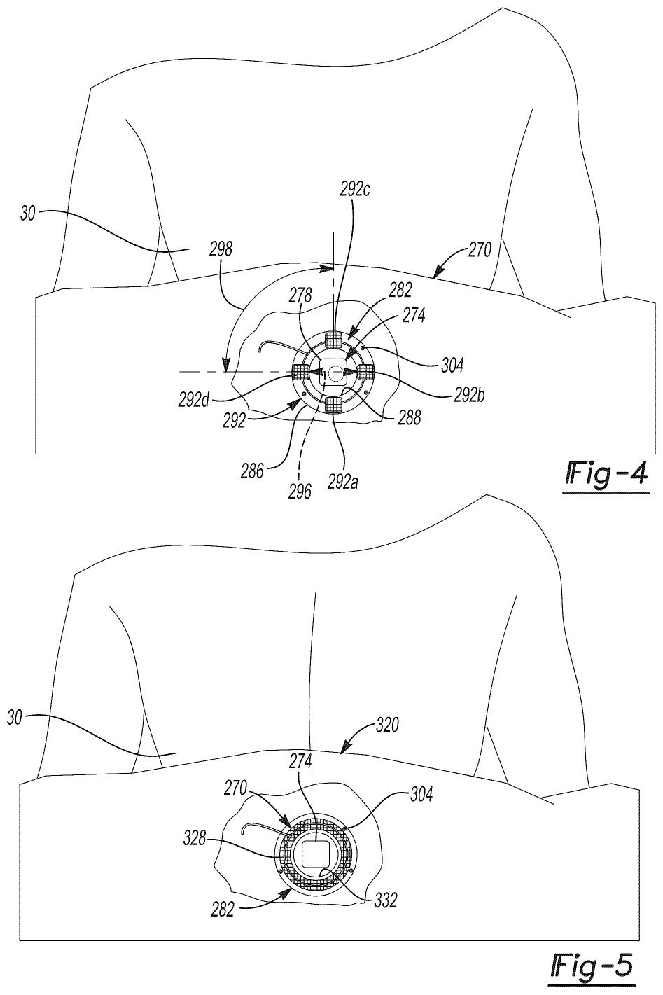

Turning to reference to FIG. 4, a surgical drape 270 is illustrated. The surgical drape 270 may be similar to the surgical drape 200 discussed above. The surgical drape 270 may include an access porthole or surgical opening 274 that is defined by one or more walls or perforations 278 through the surgical drape 270.

Positioned near the surgical access 274 may be an ultrasound window portion 282. The ultrasound window portion 282 may surround or partially surround the access 274. The access 274 may be similar to the access 204 discussed above. The ultrasound window portion may be substantially annular or toroidal including an external circumference 286 and an internal circumference 288. The ultrasound window 282 may be formed substantially similar to the ultrasound window 226 as discussed above, save for the shape of the ultrasound window 282. Thus, the ultrasound window 282 may be formed around the surgical access 274 to allow for a selected view of the subject 30 when image data is acquired with one or more ultrasound arrays 292. A selected number of ultrasound arrays 292 may be provided, such as a first ultrasound array 292a, a second ultrasound array 292b, a third ultrasound array 292c, and a fourth ultrasound array 292d. Each of the ultrasound arrays 292 may be positioned relative to the surgical access hole 274 in a selected manner, such as substantially 90 degrees of one another around the ultrasound window 282.

Again, the ultrasound arrays 292 may have known positions relative to one another, such as having a known diameter distance 296 from one another and/or that they have a selected or known angular position 298 relative to one another. Thus, each of the ultrasound arrays 292 may have known positions relative to one another. The ultrasound arrays 292 may be operated in concert to determine positions of image data relative to the subject 30 similar to the manner discussed above.

Further, the imaging system 33 may include one or more fiducial or tracking portions 304. The tracking portions 304 may be positioned relative to the ultrasound arrays 292 for tracking with the tracking system 26. Therefore, the tracking system 26 may track a position of the ultrasound array relative to the subject 30. This allows the image data may be registered to the subject 30. Again, the position of the individual ultrasound arrays 292a to 292d may be known relative to the one or more tracking devices 304. Further, the plurality of the tracking devices may be positioned on the ultrasound assembly 33, such as associating one tracking device with each of the ultrasound array portions 292a to 292d.

Turning reference to FIG. 5, a surgical drape 320 is illustrated. The surgical drape 320 may be similar to the surgical drape 270 discussed above. It may include a surgical access opening 274 relative to which an ultrasound window 282 is positioned. The ultrasound window 282 may be substantially similar to the ultrasound window 282 discussed above relative to FIG. 4. With the ultrasound window 282, however, may be an ultrasound array 324 may include a substantially similar geometry to the ultrasound window 282. One or more of the tracking devices 304 may also be provided with the ultrasound window 282.

The ultrasound array 324 may also be substantially annular or toroidal and include an external or outer circumference 328 and an internal circumference 332. The ultrasound array 324, therefore, may entirely surround the surgical access 274 and may be substantially coextensive in an annular manner with the ultrasound window 282. The ultrasound array 324, therefore, may acquire image data at any appropriate position around the surgical access window 274. As is understood by one skilled in the art, the array 324 may include a plurality of selected sizes of transducers or plurality of transmitters and plurality of receivers. Thus, the ultrasound array at 324 may include a pre-known or known geometry of the ultrasound portions of the ultrasound array 324 to determine image data relative to the access window 274. Again, the tracking portions or members 304 may also be used to track the ultrasound array 324 relative to the subject 30.

As discussed above, each of the ultrasound assemblies, including those relative to the windows 226 to 230 and the toroidal windows 282 may be formed with the respective drapes to include appropriate coupling with the subject 30. Thus, the respective ultrasound arrays may generate image data for allowing for generation or reconstruction of images of the subject 30 in a selected manner. The ultrasound arrays formed with the or in the drapes may offer a substantially close contact and precise positioning of the respective ultrasound arrays relative to the subject 30. Further, as the surgical drapes are substantially maintained during a procedure (such as on the subject 30), the ultrasound arrays may be maintained during an entire procedure in a selected position. Nevertheless, even movement of the ultrasound arrays may be determined with the navigation system 26 due to the respective tracking portions, such as the tracking portions 252, 256, 304.

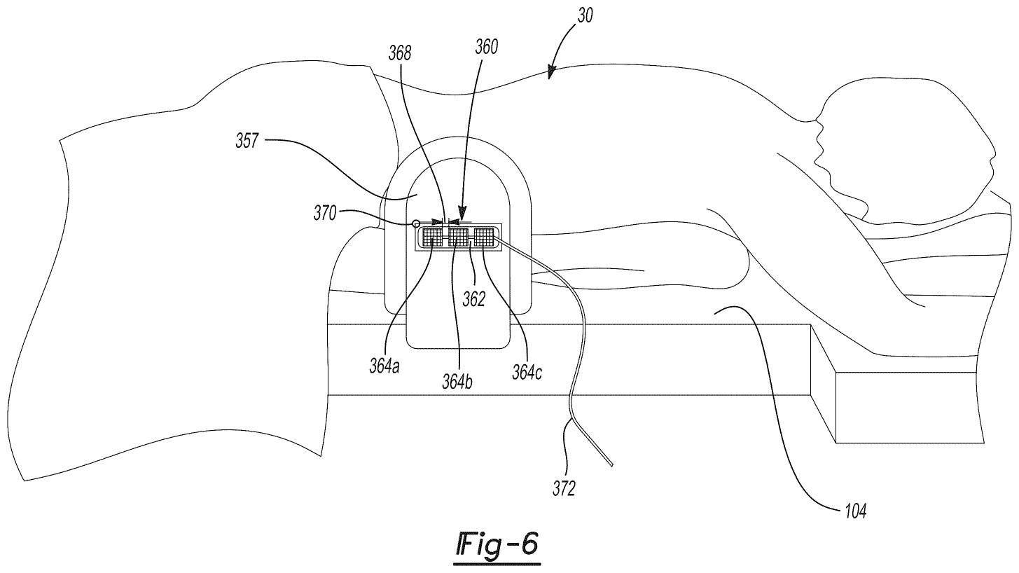

With reference to FIG. 6, the subject 30 may be positioned on a support, such as the surgical table 104 as discussed above. The surgical table 104 may have various attachments attached thereto, such as a head frame or support, a hip holder or support 357, or other appropriate portions. Regardless, the table 104, the hip holder 357, or other portions of the surgical support, such as the surgical table 104, may include or have incorporated there with an ultrasound array or imaging system 360. The ultrasound array 360 may be the imaging system 33, as discussed above. The ultrasound array 360 positioned in the hip holder or patient positioner 357 may allow the ultrasound array 360 to be positioned relative to the subject 30 for the selected portion of the procedure. This may assist in holding the ultrasound array 360 relative to the subject 30 without requiring additional support, such as an additional user or technician.

The ultrasound array 360 may include portions similar to those discussed above. For example, the ultrasound array 360 may include a window or patient contacting portion 362. The window 362 may also be a hole or passage formed in the support 357. Further, the supper 357 is merely exemplary and may be any appropriate support for the subject 30. Therefore, ultrasound waves from the ultrasound array 360 may be passed to the subject 30 to allow for imaging or collection of image data of the subject 30. The ultrasound array 360 may include various array portions, similar to the array portions 222 discussed above. The array portions may include a first array portion 364a, a second array portion 364b, and a third array portion 364c. As discussed above the various ultrasound array portion 364a, 364b, and 364c may also be transducer systems, and/or be separate transmission or receiver portions to allow for the array 360 acquire image data in any appropriate or selected manner. Nevertheless, the array portion 364 may allow for acquisition of image data of the subject 30.

The ultrasound array 360 may be provided with the window portion 362 such that to the array portion 364 are at a known position relative to one another, such as a known or fixed to distance 368 along a selected axis. Therefore, as discussed above, the imaging system portion 360 may be able to acquire image data of the subject 30 in a selected and known manner. Further, a tracking portion 370 may be associated with of the tracking array 360, such as fixed to the tracking array 360 and/or fixed to a portion relative to the tracking array. For example, the tracking portion 370 may be fixed to the hip support 357. Similarly, the imaging system 360 may be fixed to the hip support 357. Therefore, the tracking member 370 may be at a fixed position relative to the imaging array 360.

Data may be transmitted from the imaging array 360 over an appropriate connection, such as the connection 372. The connection 372 may be a wired connection, wireless connection, or combinations thereof. The connection 372 may transmit various information such as the image data, tracking information from the tracking device 370, or other appropriate information. Therefore, the connection 372 may be connected to any appropriate portion, such as the processor system assembly 102, the communication portion 110, or any other appropriate portion. Thus, the patient support, such as the table 104, may include the ultrasound array 360 to be positioned relative to the subject 30.

As discussed above, the various configurations of ultrasound assemblies, including one or more arrays, such as the US array 222, the US array 292, or the US array 364 may all include relative to one or more of the transducers thereof the various one or more tracking devices such as the tracking devices 252, 256, 304, 370, or the like. As each of the arrays may be included or positioned relative to one or more of the tracking devices, the array may be used or operated in a concerted manner. For example, to provide a collection of image data or allow generation of images of an area greater than the area imageable by any particular one of the arrays. The tracking of the tracking devices relative to one of the arrays may also allow for tracking of all of the arrays if the tracking device is positioned in a selected or known manner relative to the arrays.

For example, returning reference to FIG. 1, the plurality of arrays 222 may work together in concert. For example the arrays 222a, 222b, and 222c that are associated with the first acoustic window 226 may be operated as a unit. For example, each of the arrays 222a-222c may be positioned relative to the subject of 30 in a substantially fixed manner. Therefore, a configuration or pose of each of the arrays relative to one another may be known.

Each of the arrays may generate or acquire image data within an area or plane such as a first plane 222c′ and a second plane 222b′. The data in the plane 222c′ may be obstructed or have a shadow created by the vertebra 244. The plane 222b′ may not be obstructed by the vertebra 244 relative to a selected volume of interest or region of interest within the subject 30. Therefore, given the known configuration of the array 222b relative to the array 222c and the respective imaging planes 222b′ and 222c′, an image may be generated that reduces or eliminates any shadowing that may occur if only the array 222c′ was used to generate image data of the subject 30. Various techniques may be used to reduce or eliminate the shadowing, such as stitching or filling based upon the known positioning of the arrays and their respective imaging planes relative to the subject 30.

Therefore, an image may be generated that includes reduced or no shadowing based upon the plurality of the arrays and the respective known positions relative to one another. The known position may be based upon the configuration of the multiple arrays relative to one another, such as with the acoustic window 226. However, the two array assemblies, including those associated with the acoustic window 226 and those associated with the acoustic window 230, may also be used in combination to generate images that have reduced shadowing for other features, as discussed herein.

In a similar manner the various arrays, such as the arrays 222a, 222c and/or those associated with the first acoustic window 226 and the second acoustic window 230, may be operated to generate images or images in a plane that may be referred to as virtual or in a virtual plane. The virtual plane or virtual images may be based upon reconstructing an image or generating an image from plurality of planes. For example, the plane 222b′ and the plane 222c′ may each be respective planes generated by the respective individual ultrasound arrays 222b and 222c. A virtual plane may be generated based upon the image data acquired with the two planes 222b′ and 222c′.

For example, a plane 222x′ may be a virtual plane that that is generated between the two planes 222b′ and 222c′. The virtual plane 222x′ may be generated as if an ultrasound array was positioned between the two ultrasound arrays 222b and 222c. The virtual plane may be generated for various purposes, such as generating an image that is centered at or intersects selected anatomy or portions of the subject 30 that are not directly within a center or plane of any one of the ultrasound arrays.

Thus, the ultrasound arrays may be positioned on the subject 30 and a generative technique may be used to generate virtual planes or virtual images that are not based upon or only based upon a single one of the ultrasound arrays. Again, the arrays associated with the first acoustic window of 226 may work in concert with the arrays in the second acoustic window 230 to generate the virtual planes.

In addition to the generation of a virtual plane or virtual image, a 3D volume may be reconstructed based upon image data acquired from the multiple ultrasound arrays. As discussed above, the virtual plane, such as the virtual plane 222x′ may be generated based upon the multiple imaging planes 222b′ and 222c′ at known poses relative to one another. Also the image data acquired with the multiple ultrasound arrays, given the known poses relative to one another, allows a three-dimensional volume to be reconstructed based upon the acquired image data. As discussed above, the ultrasound arrays associated with the first acoustic window 226 may all be at known poses relative to one another, as may all of the ultrasound arrays associated with the second acoustic window 230. In addition, the tracking devices 252, 256 may be operated to determine a pose of the acoustic windows 226, 230 relative to one another and to the respective ultrasound arrays associated therewith. Thus, the image data acquired with each of the ultrasound arrays, which may be scalar ultrasound arrays, may be incorporated to generate a three-dimensional image based upon the image data acquired with all of the ultrasound arrays. The multiple scalar array may be generated based upon the known pose of each of the ultrasound arrays given the tracking and/or known poses relative to one another.

As noted above, image data may be generated of various portions of the subject 30 that may be outside of any individual one of the ultrasound arrays associated with the subject 30. The multiple arrays may operate as a unit to generate image data of the subject 30 for various purposes. As a further example, given the predetermined or known configuration of the ultrasound arrays, such as each of the ultrasound arrays 222a, 222b, and 222c associated with the single acoustic window 226, may be operated as a single multiple scalar array. One skilled in the art will understand that more than one of the US arrays may be operated in a concerted or selected manner to generate image data of the subject. Also, each of the ultrasound arrays may be understood to be a scalar array and the plurality of the arrays or the multiple arrays may be a multiple scalar array.

In addition, the ability to track each of the assemblies, such as with the tracking device 252 associated with the first acoustic window 226 and the second tracking device 256 associated with the second acoustic window 230, may allow for the two assemblies to be operated together. The two assemblies may include more than one array (i.e., more than one scalar array). Thus, two or more of the assemblies may be operated in concert as well.

As discussed above, the acoustic windows 226, 230 may be tracked with the respective tracking devices 252, 256. This may allow the two tracked imaging assemblies, including the ultrasound arrays associated with respective acoustic windows 226, 230, to have pose determined relative to one another. This may also be referred to as a registration together.

The respective tracking devices 252, 256 may be tracked relative to one another and the subject 30. Therefore, the pose of each of the ultrasound arrays 220 relative to one another may be known based upon tracking with the tracking devices 252, 256 that are at known poses relative to the ultrasound arrays 222 associated with respective acoustic windows 226, 230. This may allow the two acoustic windows 226, 230 and the respective ultrasound arrays 222 to be operated as a unit given the known configuration of all of the ultrasound arrays 222. Thus, two acoustic windows 226, 230 may be associated with the subject 30 and in the appropriate manner and the tracking devices 252, 256 may be tracked relative to the subject 30, such as relative to the subject DRF 58.

The tracking of the acoustic windows 226, 230 and/or the DRF 58 allow for a triangulation of a pose of each ultrasound array 222 relative to one another. For example, the ultrasound array 222e may be at a known pose relative to the tracking device 256. Similarly, the ultrasound array 222b may be at a known pose relative to the tracking device 252. The tracking device 252 and the tracking device 256 may be tracked relative to one another and each may be tracked relative to the subject DRF 58. Therefore, the pose of the two ultrasound arrays 222b and 222e may have known or determined poses relative to one another and the subject 30 due to the tracking of the various tracking devices 252, 256, 58. Thus the generation of image data with any of the ultrasound arrays may be known or determined relative to one another and/or any other tracking device being tracked by the navigation system 26. This allows the many ultrasound arrays to be operated as a unit given their known or determinable pose relative to one another.

In addition to the exemplary embodiment illustrated in FIG. 2, the tracking of the ultrasound array with the navigation system and/or the known poses relative to one another may be determined in any of the multi-ultrasound arrays discussed above, including those illustrated in FIG. 2 and in FIGS. 4-6. Thus, the various embodiments may be operated in a manner similar to that discussed above either separately and/or together. Therefore, for example, the ultrasound arrays including multiple ultrasound arrays may be included in any of the appropriate embodiment to generate image data according to any of the incidents discussed above.

The robotic system 20, as discussed above, may include a portion to engage an instrument 68′ and/or may include an additional robotic system 20′, as is illustrated in FIG. 2, as an optional system. According to various embodiments, the ultrasound arrays 222 may be positioned relative to the subject 30. The ultrasound arrays 222 may be used to acquire image data of the subject to allow for generation of images, as discussed above. The user 72 may manually move or manipulate the instrument 68, as illustrated in FIG. 1. Additionally, or alternatively, the robotic assembly 20′ may have an end effector 44′ to engage the instrument 68′. The robotic system 20′, therefore, may be used to move the instrument 68′ relative to the subject 30. The imaging system, including the ultrasound arrays 222, may be substantially fixed relative to the subject 30 while the robotic system 20′ moves the instrument 68′ relative to the subject 30. Thus, the robotic system 20 may move the ultrasound probe 33 and/or alternatively move the instrument 68′. Additionally or alternatively, an additional robotic assembly 20′ may be provided to move the instrument 68′. Thus, the instrument 68′ may be moved relative to the subject 30 with robotic assembly 20′ while the ultrasound arrays 222 are positioned relative to the subject, such as with the acoustic windows 226, 230. As discussed above, the robotic system 20 may move a portion of the robotic system 20, such as at least the end effector 44 relative to the subject 30.

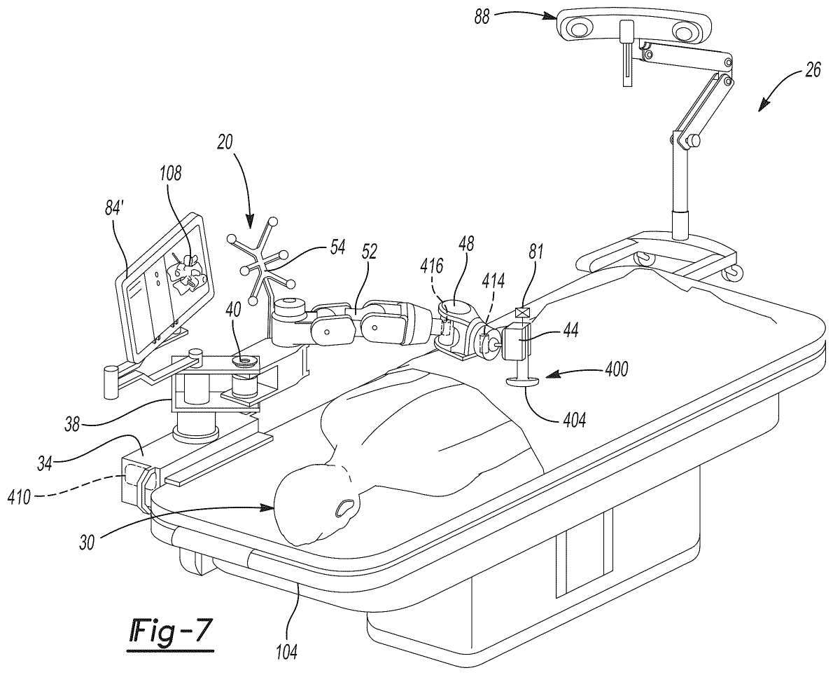

The end effector 44 may hold or be the imaging system 33, as illustrated in FIG. 1. With additional reference to FIG. 7, the imaging system 33 may be an ultrasound probe 400. The ultrasound probe 400 may include a patient contact or engagement surface 404 that may be moved along a selected portion of the subject 30, such as an exterior soft tissue surface (i.e., skin) thereof. The engagement surface 404 may include a geometry and/or feature to assist in movement and/or protect the subject 30 from pressure, a point pressure above a selected threshold. For example, a member with a smooth surface may be formed with the US probe 400 to engage the subject 30, but allow coupling of the US probe 400 with the subject 30 to acquire selected image data.

The ultrasound probe 400 may further include other portions, such as a portion to connect to the end effector 44 and/or to connect to the robotic system 20. The ultrasound probe 400 may further include a housing or other portion to which the ultrasound tracking device 81 may be connected. Therefore, the ultrasound tracking device 81 can be tracked with a selected tracking system, such as a portion of the navigation system 26 discussed above. Further, the position of the end effector 44 may be determined based upon movement of the robotic system 20, as is understood by one skilled in the art.

Generally, the robotic system 20 may be moved or operated by the user 72 and/or by executing selected instructions, such as with a processor assembly including the processor assembly 102 and/or robotic processor assembly 410. A memory portion may also be provided with the robotic assembly 20 from which instructions may be recalled and/or allowing for communication with other systems. As discussed above, the ultrasound probe 400 may be used to collect image data of the subject 30, such as with the ultrasound waves. The ultrasound probe 400, therefore, may be moved relative to the subject 32 to acquire image data of the subject 30. The robotic assembly 20 may move the ultrasound probe 400 based upon input instructions from the user72, substantially automatically based upon a predetermined imaging path and/or based upon a determination of a portion to be imaged of the subject, and/or combinations thereof. Briefly, again, the ultrasound probe 400 may include a transducer, a transmitter, and/or a receiver.

For example, the ultrasound probe 400 may be positioned relative to a selected portion of the subject 30 with the robotic system 20. The user 72 may select to have the ultrasound probe 400 moved over a selected portion of the subject 30. The selected portion of the subject may be identified by the user 72 such as using an input, including the display 84′ and/or other appropriate inputs. For example, a representation of the subject 30 may be displayed, such as based upon a general dimension of the subject 30, and the user may identify an area over which of the ultrasound probe 400 should be moved. The robotic system 20 may include for sensors such that only a maximum force is applied to the subject 30 at the contact surface 404 that contacts the surface of the subject 30. Therefore, the robotic arm 20 may move the ultrasound probe 400 in a selected manner relative to the subject 30.

In various embodiments, the robotic arm 20 may move the ultrasound probe relative to a surgical device or implant within the subject. For example, the surgical device may be tracked and its pose may be provided as a destination to be imaged and the robotic arm 20 may move according to its movement parameters to reach the destination. Also, the robotic arm could be moved relative to specific anatomy within the subject in a similar manner. Also, the destination may be manually input and the robotic arm 20 may move.

The ultrasound probe 400 may acquire image data of the subject 30, as is generally understood by one skilled in the art. The generated or determined image may then be displayed on the display device 84, such as the image 108. The image data or image generated with the ultrasound probe 400 may also be displayed in any appropriate manner and/or saved for further analysis. Thus, the ultrasound probe 400 may be moved with of the robotic system 20 to acquire image data of the subject 30.

The position of the ultrasound probe 400, which may include and/or be connected to the end effector 44, may have a position known relative to the subject 30 based upon movement of the robotic system 20. As discussed above, the robotic system 20 may have the base 34 that is fixed relative to the subject 30. The base 34 may be fixed to the patient support 104 and/or be positioned on a floor surface relative to the subject 30. Nevertheless, an origin of the base 34 relative to the subject 30 may be known and/or determined. The origin or position of the base 34 relative to the subject may be determined in any appropriate manner, such as by tracking a portion of the base 34 relative to the subject 30, tracking the robotic system tracking device 54 relative to the subject 30, or other appropriate determinations, including manual input. As a further example, the robotic system 20 may include various encoders that may determine an amount of movement of the end effector 44 relative to the base 34 to allow for determination of a position of the ultrasound probe 400 relative to the subject 30. Therefore, the position of the ultrasound probe 400, and the image data collected there with, may be known or determined relative to the subject 30. This allows the image data to be registered relative to the subject 30.

As discussed above, the ultrasound probe 400 may also be tracked with the navigation system due to the tracking device 81. The position of the ultrasound probe 400 may be known based upon the tracked position of the ultrasound probe 400 with the tracking device 81. By knowing the position of the ultrasound probe 400 relative to the subject 30, image data of selected portions of the subject 30 relative to previously acquired portions of image data may be determined and made by moving the ultrasound probe 400 with the robotic system 20. Further, the robotic system 20 may be able to precisely position the ultrasound probe 400 relative to a prior position of the ultrasound probe 400 and/or to repeat a prior position of the ultrasound probe 400. Thus, the robotic system 400 may be able to precisely place the ultrasound probe 400 relative to the subject 30 to acquire image data relative to any selected prior acquired image data and/or acquire image data at the same position as prior acquired image data.

As discussed above, imaging system, such as the ultrasound probe 400, may be moved with the robotic system 20 relative to the subject 30. The movement may be based upon a registration of the robotic coordinate system to the subject coordinate system. Also, a registration of prior acquired image data to the subject or navigation system space or coordinate system may allow or ensure that a selected region is being imaged (such as a region of interest identified in prior acquired images). In addition, or alternatively, various sensors may be included with the robotic system 20 to measure and/or ensure an appropriate amount of pressure is applied when using the ultrasound probe 400.

As understood by one skilled in the art, the ultrasound probe 400 includes the contact surface 404 that engages the subject 30. The ultrasound probe 400, including the selected transducers therein, may acquire image data of the subject 30 when at a selected or optimal position relative to the subject 30 which may include selecting or optimizing a pressure applied to the subject 30 with the ultrasound probe 400.

In various embodiments, for example, one or more sensors such as a sensor 414 and/or a sensor 416 may be included with the robotic system 20. It is understood that more than one sensor is not required, and two are illustrated as an example of various locations for the sensors. In various embodiments, the sensors 414, 416 may be positioned at various joints of the robotic system 20. Discussion herein relating to one or more of the sensors 414, 416 may be understood or relate to any appropriate sensors in the robotic system 20 that may be positioned at any appropriate portion of the robotic system 20. The sensor 414 may include a pressure sensor that may be used to sense an amount of pressure applied with the ultrasound probe 400 to the subject 30. The pressure may be measured to determine whether an appropriate amount of pressure or selected amount of pressure is applied with the ultrasound probe 400 to the subject 30.

When applying or moving the ultrasound probe 400 over selected tissue, such as stiff tissue, a selected amount of pressure may be applied to attempt to obtain or to obtain an optimal image data acquisition. Therefore, the pressure sensor 414 may be used to measure an amount of pressure applied and/or tissue resistance. A determination may be made whether additional pressure may be applied or should be applied to acquire optimal image data.

Further, as discussed above, due to the registration of the robotic coordinate system to the subject coordinate system and any prior required image data coordinate system, a determination that the ultrasound probe 400 is positioned relative to the subject over a selected heart tissue or portion (e.g., bone) may be used to assist and determine an amount of pressure to be applied. The pressure sensor for 414 may then determine the amount of pressure applied and determine whether a change in an amount of pressure is appropriate, such as by comparison of the measured pressure to a predetermined pressure. In various embodiments, for example, a greater pressure may be applied over portions of the subject not including a hard portion (such as tissue over an abdomen) as opposed to a portion over a hard portion within the subject 30. The various sensors may be used to measure a pressure being applied through the ultrasound probe 400 to the subject 30 and a comparison may be made to a predetermined pressure and/or a determination of whether additional or less pressure may be applied to achieve optimal image data acquisition.

Further, an amount of pressure being applied may be measured to determine whether deformation of tissue may be occurring. If a great amount or selected amount of pressure is applied with the ultrasound probe 400 deformation of tissues or organs may occur. The deformation may change or alter an image acquisition. For example, if a selected or greater than selected threshold pressure is applied, a position of an organ, such as a kidney or intestines, may differ from a pre-acquired image where no or little pressure is applied. Therefore, the pressure sensor 414 may be used to determine an amount of pressure applied and whether an effect to a position of an internal object, such as an organ within the subject 30, may change due to the applied pressure at the ultrasound probe 400. The pressure sensor 414 may measure the pressure and allow for a determination of whether pressure should be increased or decreased and/or whether a pressure being applied may cause a deformation or movement of an internal organ. If the pressure measured is greater than a selected threshold a determination may be made that an organ may be moved or a deformation of tissue may occur and thus the image may be analyzed or understood to possibly differ from a prior acquired image. This may also allow for acquisition of additional or alternative image data to have a different amount of applied pressure to assist in the image data acquisition with the ultrasound probe particularly when being moved with the robotic system 20.

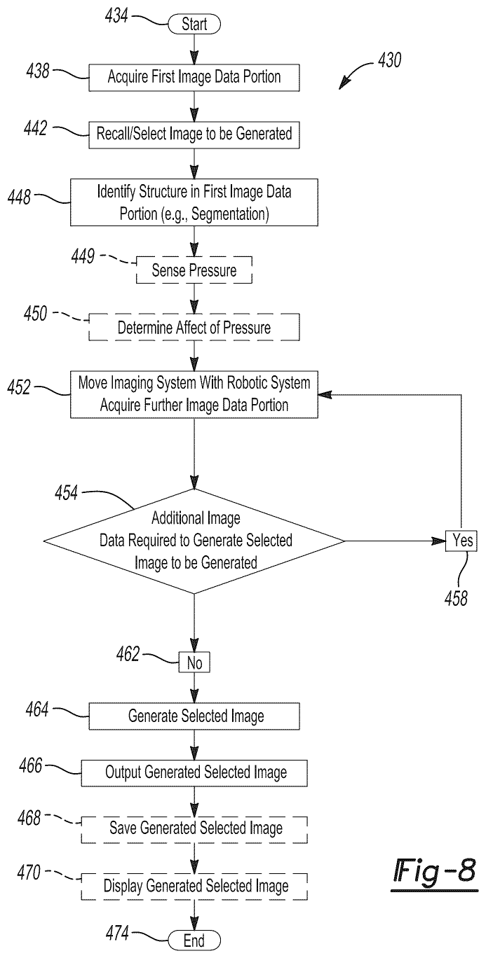

With reference to FIG. 8, a process 430 is illustrated. The process 430 may be used to generate image data of the subject 30 in a selected manner. For example, the robotic system 20 may move the ultrasound probe 400 relative to the subject 30. In moving the ultrasound probe 400 relative to the subject 30, image data may be collected at various poses (e.g., perspectives) relative to the subject 30. The various perspectives may be used in conjunction with an appropriate ultrasound probe to generate selected images of the subject 30, such as three-dimensional or volumetric images.

The process 430 may start in start Block 434. The process 430 may then acquire image data in a first position in Block 438. The image data may be acquired relative to the subject in any appropriate position. The ultrasound probe 400 may be moved by the robotic system 20 to a selected location, positioned manually by the user 72, or in another appropriate manner including combinations thereof. The image data may be acquired of the subject 30 in any appropriate manner. After acquiring image data, or at any appropriate time, a recall or selection of an image to be generated may be made in Block 442. The selection of an image to be generated may include selecting a vertebrae to image, a plurality of vertebrae, an organ of the subject, or other appropriate portion. For example, the ultrasound probe may be used to generate an image data of soft tissue relative to one or more vertebrae, image data or an image of the vertebrae, or an organ of the subject, such as the heart of the subject. Regardless in the image data after selecting the portion of which an image is to be generated an identification of a structure in the image data may occur in Block 448. The identification of the structure may include segmenting the image data acquired in Block 438. The segmenting of the image data may include finding edges or other appropriate portions in the image.

After segmenting the image, a structure may be identified. The structure in the image data may be identified as the portion or a portion of the subject to be imaged. The structure identified may be used to identify where to move the ultrasound probe 402 to acquire image data to generate image data to generate the selected image of the subject. For example, a heart wall or edge of a structure may be segmented and an identification of the structure may be made. The identification of the structure may be automatic, such as with the processor assembly 102, manually by the user 72, or in any appropriate manner. In various examples, a look up table of structures may be used for comparison to the segmented portion. Also, various machine learning systems may be trained to identify segmented features. Another example is a statistical anatomical atlas that has been generated from a population of subjects, registered to image views, and used as roadmap/guide to segment critical features within a current image.

As noted above, the robotic system 20 may include one or more sensors 414, 416 that may be pressure sensors. Thus, the process 430 may optionally include sensing or measuring a pressure based on the pressure applied with the US probe 400 to the subject 30. The sensed or measured pressure in block 449 may then be used to determine an effect of the pressure in block 450. As discussed above, the effect of the pressure may be deformation of the tissue, movement of an organ, etc. Further, the registration of the coordinate space of the robotic system 20 and the subject 30 and any prior acquired image may be compared to the sensed pressure to determining the pose of the US probe 400, such as being over or near a hard portion, such as bone. A high measure pressure may, for example, occur when the US probe 400 is being pressed onto a portion near a bone. The determination, therefore, may be to change the pressure applied, move the probe 400, analyze the acquire image data based on possible deformation, etc.

Based upon the acquisition of the image in Block 438 and the identification of the structure in Block 448 and based upon the recalled portion of the subject for which an image is to be generated in block 442, the robotic system 20 may move the ultrasound probe 400 to a subsequent or second position to acquire subsequent or second image data at block 452. For example, the ultrasound probe 400 may be moved to acquire an anterior to posterior (AP) image of the subject 30 in the block 438. A second position may include acquiring a lateral image through the heart of the subject 30. Thus, the robotic system 20 may move the ultrasound probe 400 to an appropriate position to acquire further image data.

The movement of the US probe 400 with the robotic system may be substantially automatic. For example, the robotic system 20 may be tracked relative to the subject to allow a pose of the US probe 400 to be known based n tracking the subject 30 and the robotic system 20. The US probe 400 may be automatically moved to the pose. Further, as discussed herein, portions to be imaged may be identified. The portions may be partially imaged in a first image. The system may determine further portions that required additional image data and may move the robotic system 20 accordingly to move the US probe 400 to acquire additional image data. For example, if a heart is selected to be imaged a first image may be segmented and a portion of the heart (e.g., an inferior portion of a right atrium) may be identified. The robotic system 20 may then move the US probe 400 in a manner based on known anatomy (e.g., an atlas of the heart) to acquire image data of additional portions of the heart (e.g., a superior portion of the right atrium).

After acquiring further image data, a determination may be made whether additional image data is required in Block 454. If further image data is required, a YES path 458 may be followed to Block 452 to move the ultrasound probe 400 with the robotic system 20 and acquire further image data. The process 430 may iterate to move the ultrasound probe 400 with the robotic system 20 and acquire further image data to allow for generation of the selected image.

In addition to or alternatively to the robotic arm 20 physically moving the US probe 400 and/or the contact surface 404, various beam shaping or steering techniques may be used to move the area being imaged with the US probe 400. It is understood that the beam shaping or steering may be used with any appropriate US system, including the one or more arrays 222 discussed above or according to any appropriate embodiment.

In beam forming or beam steering, various ultrasound transducer arrays may be used to generate selected beam portions, such as beam shapes or beam parameters. Selected portions of the array may be energized separately or simultaneously to generate different beam configurations and/or angles relative to a fixed position of the ultrasound probe 400. The beam steering may allow the area being imaged or where image data is being collected to change without physically moving the ultrasound probe 400. Therefore, the image data may appear to be moving and/or be imaged at a different position relative to the ultrasound probe 400 without physically moving the ultrasound probe 400. The beam steering or beam forming may be performed substantially automatically by operating or controlling the ultrasound probe 400 and the transducers therein, such as with a selected processor system as discussed above. The beam steering or beam forming may be used to collect image data at selected positions relative to the subject without physically moving the ultrasound probe 400. Therefore, image data may be acquired at different poses relative to the subject without physically moving the ultrasound probe 400.

The determination in block 454 may be substantially automatic by the system. For example, the system may include the processor 410, or any appropriate processor. The determination may be made based on a look up table regarding segmented portions of the current image, a machine learning system that is trained to determine the portions selected to be imaged, etc. In this manner, the system may determine automatically whether sufficient image data is collected.

Accordingly, if a decision is made in Block 454 that no further image data is needed a NO path 462 may be followed. The image may then be generated in block 464. As is understood by one skilled in the art, an image may be generated based upon the acquired image data with an ultrasound probe. The generated image may be a three-dimensional image, such as a three-dimensional image of a heart of the subject 30. Additionally, other image data may be included such as flow rate or pressures in the subject. Therefore, the image 108 that may be generated in block 464 may include any appropriate image data or selected data.

After generating the image in block 464, the image may be output in block 466. Outputting the image in block 466 may include various selected or optional portions, such as saving the image in block 468 and/or or displaying the generated image in block 470. As discussed above, the image 108 may be a generated image that is output in block 466.

The process may then end in Block 474. Ending the process 430 in block 474 may end the collection of image data to generate a selected image of the subject 30. A procedure may occur relative to the subject 30 based upon the acquired image data and/or generated image. Therefore, further image data may be acquired at a later time and the process 430 may be repeated. The robotic system 20 may allow for repositioning the ultrasound probe 400 at a substantially previous position of the ultrasound probe. Therefore, the robotic system 20 may move 30 the ultrasound probe 400 to the same positions of the process 430 to acquire a later or subsequent image data of the subject 30. Confirmation or subsequent image data may also be acquired with the process 430, such as for confirmation of a planned procedure and/or determining a further procedure portion to perform.

In addition to or alternatively to the above, the image data acquired in block 454 may be collected over time and the generated and/or outputted image in blocks 464 and 466 may be four-dimensional (4D) image data. The robotic arm 20, according to various embodiments, may be moved to acquire the 4D image data. Creation of 4D data may be for a cardiac use case where the heart is moving. In such an instance image data or an image may be 3D and it may change over time, thus 4D data. The 4D image data may be displayed, such as a movie. In various embodiments, the 4D image data and/or image may be segmented. The Segmented image may be registered to preoperative multiphase (4D) cardiac CT or other appropriate images. Thus, the generated image may be a 4D image and it may be registered to other 4D images.

In this manner, the robotic control system may, such as by executing instructions with a processor, to move the ultrasound transducer system to acquire image data to generate a 4D image data and/or image of the subject. The acquired 4D image data and/or image may also be segmented and may be registered to the subject and/or preacquired images such as for identification of selected structures, or verification of a predetermined outcome, etc.

As discussed above, the imaging system 33 may include an ultrasound imaging probe, which may include a housing that is moved relative to the subject 32 to acquire image date of the subject 30. The image data may be used to generate and display images, such as the image 108. The image that is displayed may, however, be based upon the position of the ultrasound probe relative to the subject 30. The user, particularly if the ultrasound probe is manipulated by a robotic system, such as the robotic system 20, may select to have a particular area or region imaged substantially continuously. The system may determine movements of the ultrasound probe to position the probe relative to the subject to maintain the selected image for displaying to and by the user 72. That is, ultrasound images may generally be collected to display substantially in real time. Further, the user 72 may identify to have a portion of an instrument, for example at the tracked instrument 68, to be maintained within a portion of the image that is generated by image data from the ultrasound probe. Thus, with reference to the above, and the below flowcharts, a system may be used to determine a portion of the subject that is imaged to maintain the selected image, determine a position of an instrument and ensure that a portion of the instrument is within the image, and substantially automatically moving the ultrasound probe to maintain the selected image or instrument within the image. Additionally or alternatively, the imaging system 33 may be fixed or frozen at a selected pose once achieved, as discussed herein.

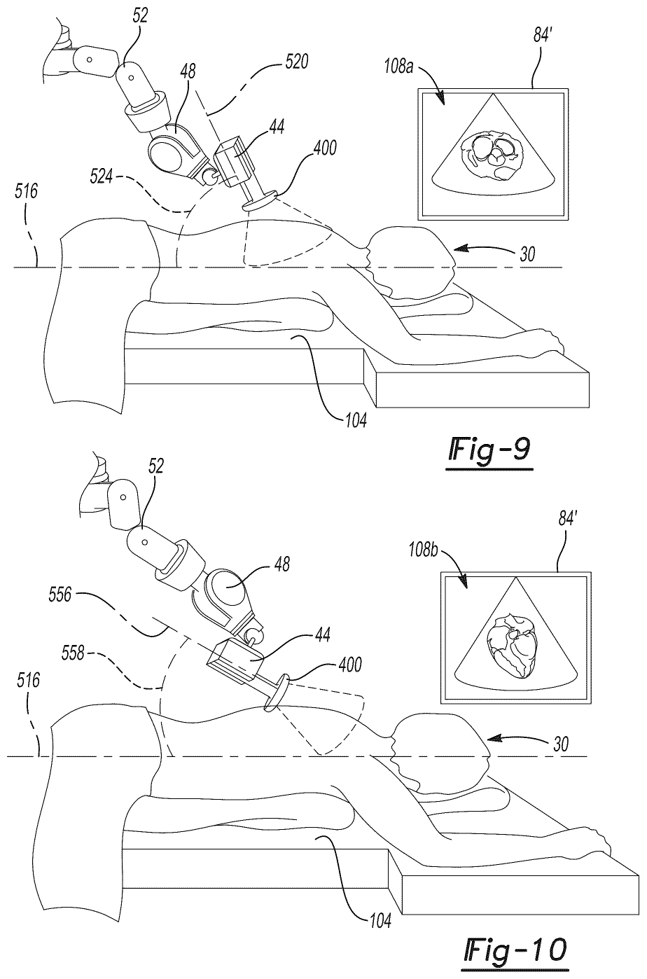

With initial reference to FIG. 9 and FIG. 10, the robotic system 20 may position the ultrasound probe 400 relative to the subject 30. The subject 30 may be positioned in any appropriate portion, such as on the table or support 104. The ultrasound probe 400 may be moved relative to the subject 30 in any appropriate manner, such as in selected spatial locations (including three degrees of freedom) and orientations (including selected, such as three, degrees of freedom) relative to the subject 30. Therefore, the ultrasound probe 400 may be moved in selected degrees of freedom, which may be limited by the robotic system 20, or include any appropriate degree of freedom of motion, such as six degree of freedom including three degree of freedom in location and three degree of freedom in orientation. The robotic system 20 may move the ultrasound probe relative to the subject to acquire various images, such as a first image 10a, as illustrated in FIG. 9, or a second image 108b is illustrated in FIG. 10. The image 108a, 108b may be displayed on the respective displays, such as a display 84′. The images 108a, 108b may be displayed on the display is 84′ based upon a position of the ultrasound probe 400 and/or the anatomy of the subject 30. It is understood, however, that the ultrasound probe 400, as the imaging system 33, may image any appropriate subject and the human subject is merely exemplary.

Nevertheless of the images 108a, 108b may be images of a heart of the subject that may be in selected phases. For example the image 108a may illustrate and anterior-to-posterior view of the heart of the subject 30 and/or a systole phase of the heart. The image 108b may be a lateral- or superior-to-inferior view of the heart and/or a display of the heart in a diastole phase. It is understood, however, that any appropriate portion of the subject 30 may be displayed with the selected image. Further, the portions of the subject may have phases that include or change in physical dimensions or geometry of portions within the subject over time, such as rhythmically. One skilled in the art understands that the heart phase changes the shape of the heart over time in a substantially rhythmic manner. Other portions of the anatomy of the subject 30 may also include a similar changes.

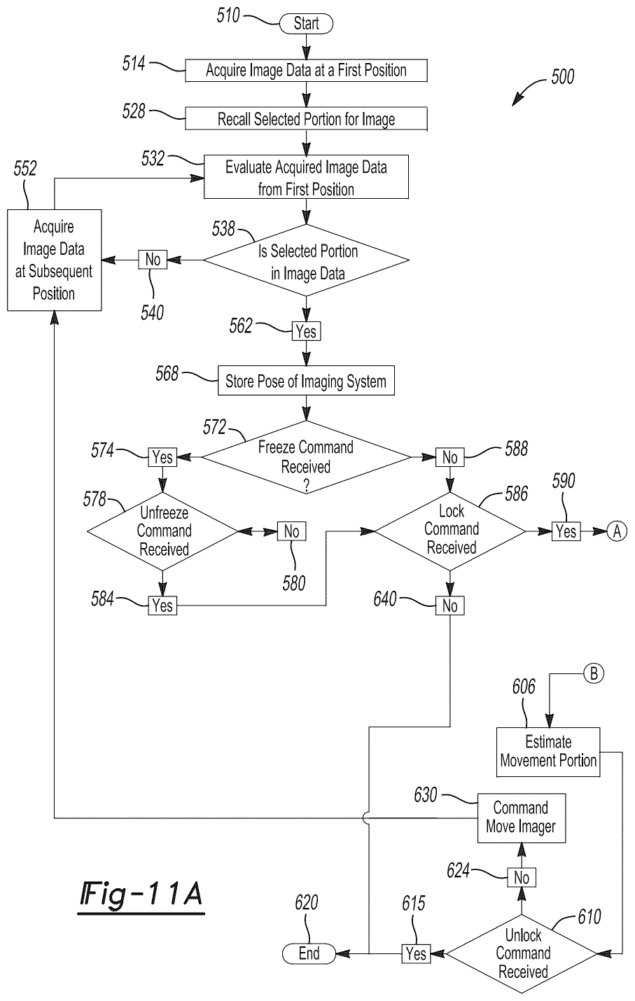

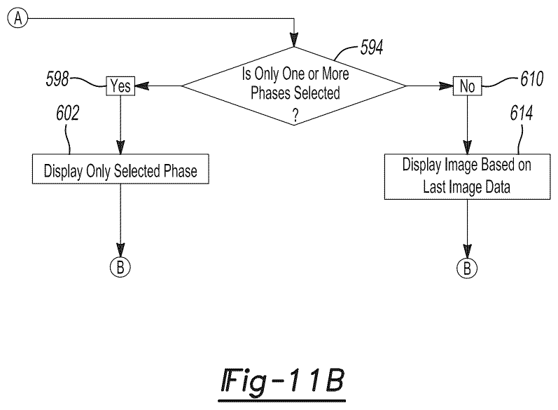

With continuing reference to FIGS. 9 and 10, and additional reference to FIG. 11, a process 500 is illustrated. The process 500 is a process for moving the imaging system, including the ultrasound probe 400, relative to the subject 30 to generate image data of the subject 30. The process 500 also allows for ensuring that a selected portion of the subject is maintained in the image generated with the image data from the ultrasound probe 400. As discussed above and is understood by one skilled in the art, the ultrasound probe 400 as the imaging system 33 may collect or acquire image data of the subject 30. The image data may be analyzed, evaluated, and processed to allow for generation of images, such as the image is 108a, 108b. The process 500 may be incorporated in instructions that are executed by any appropriate processor, such as the processor 410. The instructions may be formulated into an appropriate algorithm.

The process 500 may begin in start Block 510. The start Block 510 may be any appropriate starting portion of the process 500, such as the user 72 initiating use of the ultrasound probe 400 that may allow for analysis and/or evaluation of the image data. The image controller 96 may include a processor and may process image data from any appropriate imaging system, such as the imaging system 80, or the imaging system including the ultrasound probe 400. Accordingly, acquisition of image data at a first position may be made in Block 514.

Acquiring image data at a first position may include acquiring image data in the position is illustrated in FIG. 9. The ultrasound probe 400 may be positioned at a first position and orientation relative to the subject 30. For example, the subject 30 may extend along a long axis 516. The ultrasound probe 400 may be positioned by the robotic system 20 along an axis 520. Therefore, the ultrasound probe may be at a first angle 524 relative to the long axis 514 in a first position. Image data may be acquired at any appropriate rate with the ultrasound probe 400, such as at a selected frame rate and/or over a selected period of time.

At any appropriate time, such as after acquiring the image data at the first position in Block 514, a recall of a selected portion to be imaged or to be in the image may be made in Block 528. The recall of a selected portion for the image may be any appropriate recall, such as to ensure that a selected portion, such is a right atrium, is in the image. The controller may access a memory and/or the user 72 may input the selected portion to be in the image. The image data acquired in Block 514 may be evaluated in Block 532.

The evaluation of the image data in Block 532 may be appropriate processing and/or evaluation. For example, the image data may be segmented or analyzed for various features. Further the image data may be normalized or otherwise processed for further processing in the process 500.

A determination of whether the selected portion is in the image is made in Block 538. The determination in Block 538 may be based upon various appropriate procedures. According to various embodiments, the user 72 may view the image and determine whether the selected portion is present in the image. The evaluation of the acquired image data in Block 532 may also include generating and displaying an image based upon the acquired image data. The determination of Block 538 may also include the execution of selected algorithms or instructions based on algorithms such as comparison to look up table, comparison to a predefined atlas, a spline analysis, or edge detection and shaped detection in the image data. The selected algorithms may identify various features and determine whether they match the selected portions recalled from block 528. Further, machine learning systems may be used to analyze the image data to determine whether the selected portions are present in the image. Regardless, a determination Block 538 may be made as to whether the selected/recalled portion to be imaged is in the image data.

If the selected portion is not in the image data, a NO path 540 may be followed to acquire new image data in subsequent positions in Block 542. Image data acquired at subsequent positions may be image data in addition to the image data acquired of the first position. Thus, the ultrasound probe 400 may be used to acquire additional data. For example, as illustrated in FIG. 10, the ultrasound probe 400 may be positioned relative to the long axis 516 of the patient at a second axis 556. Thus, the ultrasound probe axis 556 may be at a second angle 558 relative to the long axis 516 of the subject 30. The image data acquired at the subsequent position may be the subsequent a position as illustrated in FIG. 10.

After acquiring the subsequent image data at the subsequent position, the process may loop to the evaluation Block 532. The process 500, therefore, may loop to ensure that the acquired image data includes the selected portion. A user may use the ultrasound probe 400 to acquire any appropriate image data at any appropriate position. The ultrasound probe 400 may be moved to selected random positions with the robotic system 20 and/or based upon inputs from the user 72. For example, the robotic system 20 may move in a grid search pattern relative to an initial position. Further, as discussed above, the ultrasound probe 400 may be held by the robotic system 20 that is tracked with the navigation system and the subject 30 may also be tracked with the patient tracker 58. Thus the portion to be imaged may be determined relative to the patient tracker 58 and the probe 400 may be moved relative thereto to acquire image data and/or a search pattern may be made relative to the initial determined position.

If a determination is made that the selected portion is in the image data, a YES path 562 may be followed. In following the YES path 562, the pose of the imaging system may be stored in Block 568. Determining the pose of the imaging system may be performed in various manners. As discussed above, the controller 96 may include a processor and/or memory. A pose of the imaging system may be stored in the controller. The pose may be based upon a navigational tracked pose of the imaging system, including the ultrasound probe 400. The tracking device 81 may be positioned on the probe 400 and tracked to determine a pose of the probe 400. Additionally or alternatively, the robotic system 20 may include an encoder to determine a pose of the ultrasound probe 400 and/or the end effector 44. The pose of the end effector 44 may be saved and selected portions and/or accessed with the robotic processor 410. Regardless, the pose of the imaging system may be stored in Block 568.

A determination of whether a freeze command has been received may be made in Block 572. The freeze command may include commanding the ultrasound probe 400 to be maintained at the pose stored in Block 568. By freezing of the ultrasound probe 400 at the stored pose, the image acquired of the subject 30 may be maintained relative or based upon the stored (e.g., last) pose of the ultrasound probe 400. For example, the ultrasound probe may be positioned at the position is illustrated in FIG. 9, and a freeze command may be made to ensure that the ultrasound probe stays at that pose. This pose may be the pose which includes the selected portion, as discussed above.

If a freeze command has been received, a YES path 564 may be followed to determine whether an unfreeze command has been received in Block 578. If an unfreeze command has not been received, a NO path or loop 580 may be followed. The NO path loop 580 ensures that the ultrasound probe 400 is maintained at the freeze command pose, which includes the stored pose from Block 568. Thus, once frozen, the ultrasound probe 400 will maintain its pose relative to the subject 30 or other appropriate selected portion, until unfrozen. A command to unfreeze may then follow a YES path 584 to determine whether a lock command has been received in Block 586. Similarly, a NO path 588 may be followed to the a lock command received determination when a freeze command has not been received in Block 572. Therefore, a determination of whether a lock command has been received in Block 586 may be reached based upon the two paths as discussed above.

The process 500 may also make a determination of whether a lock command has been received in Block 586. Briefly, the lock command locks the ultrasound probe 400 image data acquisition pose to ensure that a selected view is maintained. For example, is illustrated in FIG. 10, the ultrasound probe is positioned to generate image data to generate the image 108b. While the ultrasound probe 400 may be frozen to ensure that the ultrasound probe 400 is not moved over time relative to another position the lock command maintains a selected view. As discussed above, a recalled selected portion to be viewed or in the image may be made in Block 528. The lock command may ensure that the ultrasound probe 400 maintains that view, such as of the heart of the subject 30 in the image regardless of movement of the heart or the subject. Therefore, the image 108b may be maintained regardless of movement of the subject 30, movement of portions relative to the subject 30, or even movements of the ultrasound probe 400. The system may operate, substantially automatically, to maintain the selected view, such as the image 108b.

Accordingly, if a lock command is received, a YES path 590 may be followed. The YES path 590 may then follow to a determination of whether one or more poses is selected in Block 594. It is understood that the selection of one or more poses is optional, and therefore the selection is not required in the process 500. Nevertheless if a selected pose is selected and determined in Block 594 a YES path 598 may be followed to display only the selected phase in Block 602. The selected phase may be the one selected or recalled in Block 594 and/or in Block 528. For example, the diastole or systole phase of the heart may be selected to be displayed. Even if the imaging system moves over time, and/or remains in a selected position to ensure a selected viewing image, the phase of the heart may change. While image data is collected with the ultrasound probe 400 over time, only a selected phase image may be displayed, such as one of the phases or a selected phase of the heart. The system may be operated or commended to display only the selected phase in Block 602 and the process 500 then may continue to estimate movement of a portion in Block 606.

If a selected phase is not selected in Block 594, however, a NO path 610 may be followed such that an image that is displayed is simply the last or selected image in block 614. Therefore, as discussed above, the ultrasound probe 400 may collect image data substantially in real time and continuously. Therefore, the image 108b may be the image that is last collected with the ultrasound probe 400. For example, the ultrasound probe 400 may collect image data at about 30 frames per second. Therefore, the image 108b is the last image frame collected with the ultrasound probe 400. Again, following the display of the image of Block 614, the process 500 may continue to the estimate movement of the portion in Block 606.

The process 500 may continue with an estimation of a movement of the selected portion, such as based upon the subject motion, instrument motion relative to the portion, or other features. For example, the image data may illustrate that the selected portion is being acted upon via an instrument, such as for resection, implantation, or the like. A possible or average motion may be used to estimate a movement of the portion. Further the system may recall predetermined or known motion relative to a portion of the subject, such as due to a beating of the heart, to estimate a motion of the selected portion. Therefore, the system in the process 500 may estimate a movement of the portion in Block 606.

Following the estimated movement of the portion, an unlock command received determination may be made in Block 610. If an unlock command is received, a YES path may be followed in Block 615. If the imaging system, including the ultrasound probe 400, is commanded to not lock onto any particular image portion and may be moved and/or maintain a selected position. Therefore, if the unlocked command is received, the YES path 615 may be followed to end the process in Block 620. The end of the process 500 will be discussed further below.

If an unlock command is not received, a NO path 624 may be followed. The NO path 624 may then move or reach a command to move imager in Block 630. The command to move the imager may include moving the imager in any appropriate manner and to move the imager, including the US probe 400, to maintain the selected image based upon the estimated movement of the portion from block 606. Therefore, based upon the maintaining of the lock command, a move command may be made. The move command may include a movement of the robotic system 20 in a selected three-dimensional space location and/or three-dimensional orientation for movement of the imaging system 400. The movement command may include a selected movement in any appropriate axis and/or orientation to position the imaging system, including the ultrasound probe 400, relative to the subject 30 to maintain the imaging of the selected portion. After the move command is made, the imaging system may be moved and an acquisition of image data at a subsequent position may be made in Block 552. The movement in block 552 may include beam steering and/or forming as discussed above. Therefore, the process 500 may loop or iterate to maintain acquisition of image data of a selected portion of the subject, such as the locked on feature in the subject 30.

Returning reference to the decision Block 586, a lock command received may follow a NO path 640. The NO path 640 may also go to the end Block 620. Therefore, if no lock command is received, the process may end in Block 620 allowing the imaging system to acquire images as selected. In this manner, the user 72 may manually move the ultrasound probe 400 and/or manually command the robotic system 20 to move the imaging system, including the ultrasound probe 400. This allows the ultrasound probe 400 to be moved in any appropriate manner to achieve image data of the subject 30 as selected by the user 72.

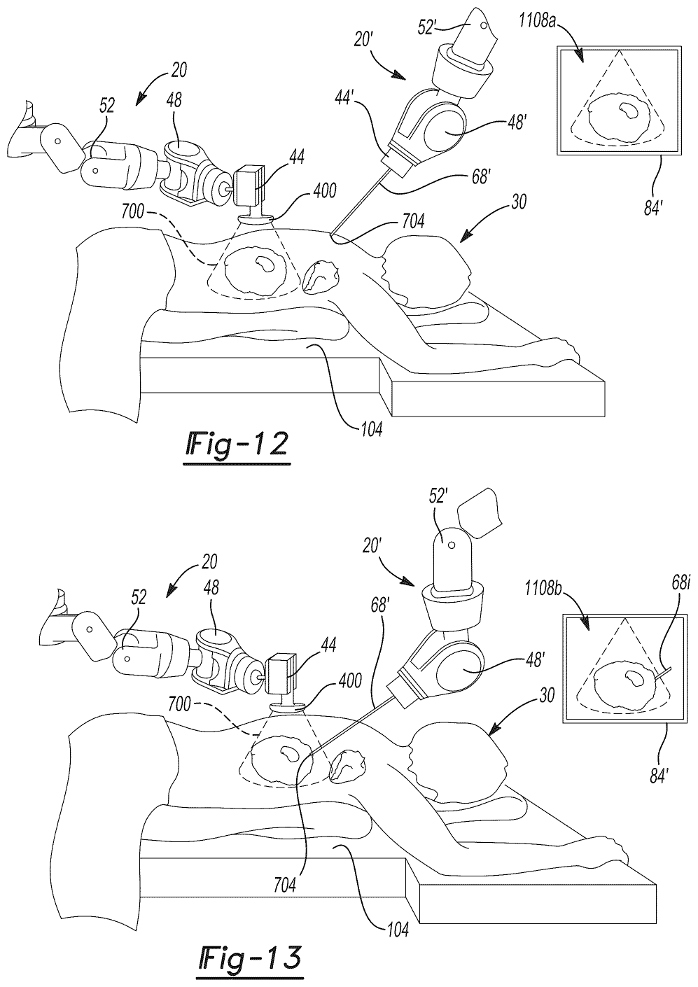

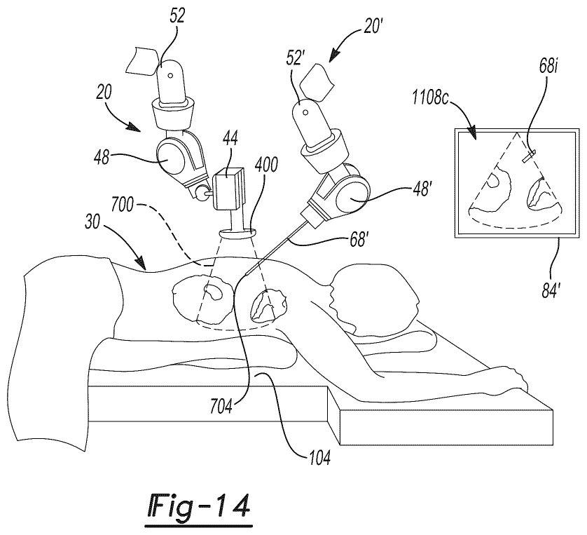

In addition to imaging selected features of the subject 30, such as various portions of the anatomy, including the heart, therein, the ultrasound probe 400 may be used to image instruments or portions that are moved relative to the subject 30. For example, is illustrated in FIG. 12, FIG. 13, and FIG. 14, the ultrasound probe 400 may move with the robotic system 20 relative to the subject 30. The ultrasound probe 400 may acquire image data of various portions of the subject, as discussed above. Various images may be displayed on the display device 84′ and/or any appropriate display device, based upon the acquired image data. Images 1108a, 1108b, and 1108c are illustrated in FIGS. 12-14. The images 1108 may be similar to the image 108, as discussed above. However, the images 1108 are discussed herein for clarity relative to the following discussion.

The ultrasound probe 400 may be moved to acquire image data at various positions relative to the subject 30 to allow for a generation or reconstruction of the images 1108. An instrument 68′ may be moved relative to the subject 30. The instrument 68′ may be substantially similar to the instrument 68, discussed above. According to various embodiments, the instrument 68′ may be held and/or moved with a robotic system 20′. Robotic system 20′ may be substantially similar to the robotic system 20. The system 20′ may include an end effector 44′ that engages the instrument 68′. Therefore the instrument 68′ may be moved with the robotic system 20′ to various positions relative to the subject 30. The position of the instrument 68′ may be tracked, according to various embodiments. As discussed above the navigation system may track a tracking device associated with the instrument 68′. In addition and/or alternatively the robotic system 20′ may have a position known relative to the robotic system 20 and/or the subject 30 to allow for tracking or knowing a position of the instrument 68′ relative to the subject 30. Thus, according to various embodiments (including those otherwise disclosed and not only illustrated in FIGS. 12-14), the robotic system 20 may hold and or position (e.g., move) the US probe 400 separate from and/or while the robotic system 20′ may hold and position (e.g., move) the instrument 68′. In various embodiments, however, the robotic system 20 moving the ultrasound probe 400 may have a position unknown relative to the robotic system 20′ moving the instrument 68′. However, the robotic system 20 may move the ultrasound probe 400 to capture an image of at least a portion of the instrument 68′ and it may also move the ultrasound probe 400 to maintain at least a portion, including a selected portion, of the instrument 68′ in the image 1108 generated with image data from the ultrasound probe 400.

Briefly, as illustrated Fig. FIG. 12, the ultrasound probe 400 may capture or acquire image data from a region 700 of the subject 30. The region 700 may not include a portion of the instrument 68′, such as a distal tip 704 of the instrument 68′. Turning reference to FIG. 13, the ultrasound probe 400 and/or the instrument 68′, may be moved to image a region 700 of the US probe 400 and/or encompasses at least the distal tip 704 of the instrument 68′. Therefore, the image 1108a-c may include an icon or graphical representation of the instrument 68′ as the graphical representation 68i′. As discussed further herein, according to various processes or methods, the ultrasound probe 400 may move to a second position or different position relative to the subject and/or the instrument 68′ may move relative to the subject such that the image area 700 of the ultrasound probe 400 continues to ensure that at least the distal tip 704 of the instrument 68′ is within the volume being imaged 700 and included in the image 1108c. Therefore, the image 1108c may include the graphical representation 68i′ of the instrument 68′, including the distal and 704 thereof.

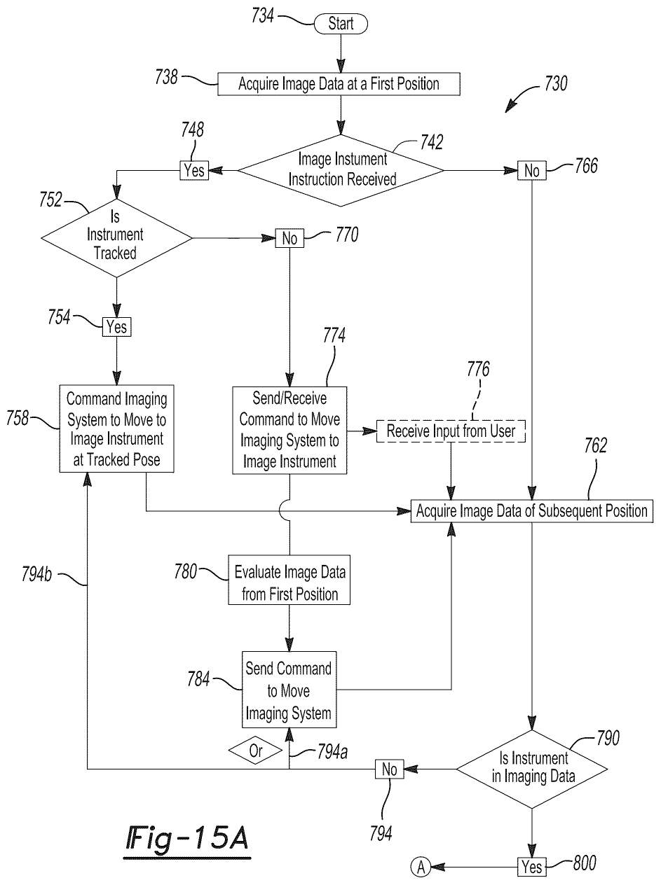

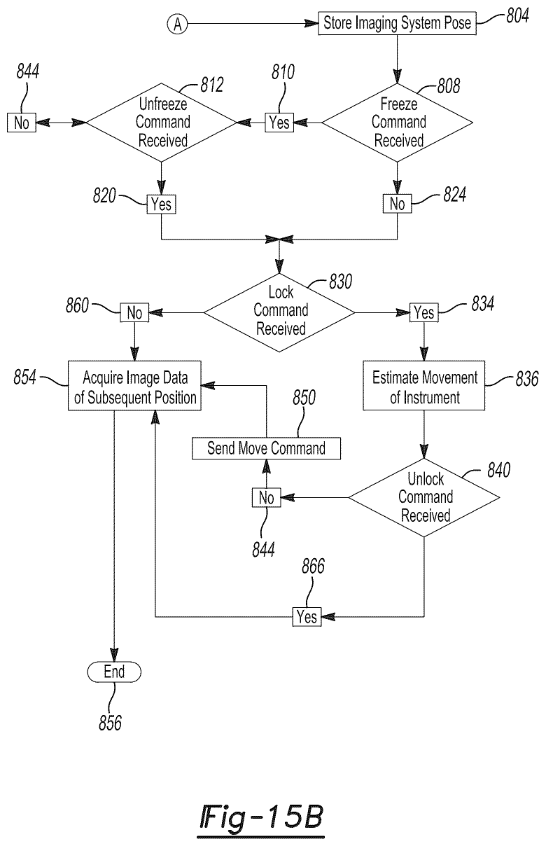

Turning reference to FIG. 15, the system may execute a process 730 to track or illustrate a portion of an instrument, such as the instrument 68′, in the image 1108. The process 730 may include portions that are similar to the process 500, discussed above. Those portions will not be discussed in detail herein. Nevertheless, the process 730 may be used to generate the image 1108 that includes at least a portion of the instrument 68′, such as the distal tip 704. The image 1108, therefore, may be illustrated to assist the user 72 in understanding the pose of the instrument 68′ relative to the subject 30 and/or other portions within the image 1108. For example, the instrument 68′ may be moved relative to the subject 30 to assist in performing a procedure on selected portions of the patient, such as a liver, the heart, or the like. The instrument 68′ may be moved by the robotic system 20′ and/or the instrument 68 may be moved by the user 72 regardless, the imaging system including the ultrasound probe 400 may be moved to maintain a selected portion of the instrument 68, 68′ within the image 1108. The process 730 may be incorporated in instructions that are executed by any appropriate processor, such as the processor 410. The instructions may be formulated into an appropriate algorithm.

Initially, the process 730 may start in start Block 734. The process 730 may then acquire image data at a first position in Block 738. As discussed above, the first image data may be any appropriate image data and acquired in any position that may be understood or labeled as the first position. Nevertheless, the imaging system 400 may be moved, as discussed further herein.

At any appropriate time, such as before or after acquiring the first image data, an instruction of whether an instrument is to be imaged may be received in Block 742. Similar to the determination or receiving an input two image selected portions of the anatomy of the subject, or any appropriate portion of the subject, the instruction to image a selected portion or to ensure that a portion of the instrument is in the image may be received in Block 742.

If an instruction has been received, a YES path 746 may be followed. The YES path may then follow to a decision block of whether the instrument is tracked in Block 752. As discussed above, the instrument 68, 68′ may be tracked with various systems. For example, the navigation system may be used to determine the pose of the instrument 68′. Additionally, as noted above, the robotic system 20′ may move the instrument 68′. The robotic system 20′ may be tracked with the navigation system and/or understand its pose based upon the various systems of the robotic system, such as in encoders thereof. Therefore, the instrument may be tracked, according to various embodiments. If the instrument is tracked a YES path 754 may be followed.

If the instrument is tracked a command to move the imaging system to the tracked pose of the instrument may be sent in Block 758. The command to move the imaging system may be a command to move with the imaging system to a selected position and/or orientation in three-dimensional space relative to the subject 30. For example, the instrument 68, 68′ may be tracked relative to the subject 30. It is understood, however, that the coordinate system of the robotic system 20 that holds the ultrasound probe 400 may be registered or coordinated with any other appropriate coordinate space to allow the command to move the robotic system 20 to move the ultrasound probe 400 to any appropriate pose.

The command may be to move the robotic system 20 to move the ultrasound probe 400 to any appropriate pose. At the appropriate pose, based upon the command to move the imaging system including the ultrasound probe 400, the ultrasound probe may acquire subsequent image data in Block 762. The additional image data may be selected to include or attempt to include a selected portion of the instrument, if the instructions are received as discussed above. Regardless, the acquisition of subsequent image data will be image data that is collected after an initial or prior image data, as is understood by one skilled in the art.

The process 730 may include various other paths, including a path if an instrument is not to be imaged, then a NO path 766 may be followed. The NO path 766 may also follow to acquire additional or subsequent image data in Block 762. Therefore, if no instruction are received to image an instrument, the imaging system, including the ultrasound probe 400, may be used to acquire any appropriate subsequent image data such as manually selected by the user 72, automatically selected by the system, or to image a selected portion of the anatomy or subject 30 as discussed above.

Further, if the decision Block 752 identifies or includes that the instrument is not tracked, a NO path 770 may be followed. If the instrument is not tracked, a send and/or receive command to move the imaging system to image the instrument may be made in Block 774. The movement in block 774 may include beam steering and/or forming as discussed above. If a user inputs a direction or a command, an optional receive input from user maybe followed in Block 776. The receive input from user may include the user identifying a direction or amount of movement of the robotic system 20 to move the ultrasound probe 400. Alternatively, the command may be sent to move the robotic system 20 in any appropriate or selected manner (e.g., a planned search or movement pattern) to image the subject. In this way, the ultrasound probe 400 may be moved based upon instruction from a user, such as the user 72. Thus, it is understood that the ultrasound probe 400 may be moved based upon a manual input, as is understood by one skilled in the art.

Additionally or alternatively, an input or command may include an evaluation of image data at the first position in Block 780. The evaluation of the image data at the first position may include an evaluation of the type of instrument 68′, a selected portion of the anatomy or identification of the anatomy, or the like. For example, if the evaluation of the image data at the first position evaluates that the portion being imaged includes the heart, the system may recall or understand a beating or cyclic motion of the heart and possible movements thereof to determine a future position of the instrument (e.g., such as heart motion moving the instrument). Further the image data may be evaluated to determine a possible movement of the instrument based upon the type of instruments, and/or a predetermined plan. For example, the system may segment the image data and identify that the instrument is an ablation probe. Therefore, the system may recall a movement portion to be ablated and understand or determine a possible future movement of the instrument 60′. Regardless evaluation of the image data at the first position may be made to select or provide a guess of a future position of the instrument.

Therefore, based upon the evaluation of the image data a command to move the imaging system, such as moving the robotic system 20, may be generated and/or sent. Based upon the selected command to move the imaging system, the imaging system including the ultrasound probe 400 may be moved. Following the send command and/or movement of the imaging system 400, the imaging system may acquire additional image data or subsequent image data in Block 762. This allows the imaging system to acquire further image data to attempt to image the instrument as noted above. Thus, the imaging system including the ultrasound probe 400 may be moved based upon various inputs and/or analyses to acquire subsequent image data.

After the acquisition of subsequent image data in block 762, following any of the above-noted pathways, a determination of whether an instrument is in the subsequent image data may be made in Block 790. The determination of whether the instrument is in the image data may be based upon various appropriate processes. For example, the image 1108 may be displayed and the user 72 may identify or confirm that the instrument is in the image data. Further, various image analyses may occur. For example, segmentation of the image data may occur and a comparison of the image data to a look up table may be made to determine whether or not the instrument is in the image. Further, according to various embodiments, a machine learning system may have been trained to recognize the instrument 68, 68′. Therefore, the process or system may execute the machine learning system to evaluate and/or confirm that the instrument is in the image.

Regardless, the determination Block 790 may determine whether or not to the instrument is in the image data. If the instrument is not in the image data a NO path 794 may be followed. A No path may follow at least two processes, a first path 794 to send a command to move the imaging system in Block 784, as discussed above. An alternative path may include commanding the imaging system to move in Block 758. Therefore, the NO path 794 may include a path based upon prior instructions, such as whether the imaging system is to identify or move based upon a manual input and/or based upon an image analysis.

If the determination in Block 794 is that the instrument is in the image data, a YES path 800 may be followed to store the imaging system pose in block 804. The imaging system pose that is stored in Block 804 may be the imaging system pose similar as noted above in Block 568. The pose of the imaging system may include the position of the imaging system, such as the ultrasound probe 400, relative to the subject 30. The imaging system pose may include a pose of the imaging system, such as the ultrasound probe 400, in any appropriate coordinate system. Regardless of the position of the imaging system may be stored in Block 804.

A determination of a freeze command in block 808 may then be made. If a freeze command is received in Block 808, a YES path 810 may be followed. The YES path 810 may follow to a determination of whether an unfreeze command has been received in Block 812. As discussed above, the freeze command may include freezing the imaging system, such as the ultrasound probe 400, at a selected position in space. Therefore, the freeze command may include freezing or ensuring that the ultrasound imaging probe 400 does not move. If an unfreeze command is not received, a NO loop path 814 may be followed. The NO loop command 814 may loop the determination block of whether unfreeze command is received until unfreeze command is received, if a freeze command is first received in Block 808. If an unfreeze command is received, a YES path 820 may be followed.

If a freeze command is not received, a NO path 824 may be followed. Therefore, the no freeze command or the unfreeze command may be followed to a determination of whether a lock command is received in Block 830. If a lock command is received, a YES path 834 may be followed. A lock command 830 may be similar to the lock command discussed above in Block 586. Therefore, if the instrument is imaged or selected to be imaged, the lock command may include determining or ensuring that the portion of the image includes the instrument 68, 68′.

Accordingly, if the lock command received follows the YES path 834, an estimation of movement of the instrument may occur in Block 836. The estimation of movement of the instrument may include an estimation of motion of the instrument 68, 68′. The estimate may include a recall from a selected Lookup table of whether the instruments would move a large amount or a small amount. Further, the estimate of movement may include receiving information from the robotic system 20′ regarding movements of the robotic system 20′ that holds or moves the instrument 68′.

After estimating movement of the instrument, a determination of whether an unlock command is received may be made in Block 840. The unlock command may include unlocking the imaging system, such as the ultrasound probe 400, from imaging the instrument 68, 68′. If the unlock command is not received a NO path 866 may be followed to send a move command to the imaging system in Block 850. The move command may be based upon a selected or determined movement of the instrument or an estimated movement of the instrument. The move command may be made to the robotic system 20 that holds or moves the ultrasound probe 400. The move command may include an amount of movement or a final a position and/or orientation of the ultrasound probe 400 to which the robotic system 20 may move the US probe 400. The amount of movement and/or a final position of the ultrasound probe may be made as the move command. The move command a be sent to move the ultrasound probe 400, or any appropriate imaging system such as the imaging system 33.

Subsequent image data may then be acquired in Block 854 after movement of the US probe 400. The process may then end in Block 856. Ending the process 730 in end block 856 may include any appropriate ending procedure. The ending the process may include stopping automatic movements of the ultrasound probe 400. Ending the procedure in block 856 may also include a saving or and/or displaying selected image data, such as image data acquired with the ultrasound probe 400. Regardless any the procedure may end in Block 856 may include ensuring that image data is acquired of selected portions, including subsequent or later positions which may selectively include the instrument 68, 68′.

If a lock command is not received, a NO path 860 may be followed to acquire subsequent image data in Block 854 and further end in block 856. Again ending the procedure in block 856 may be similar to that discussed above. Also if an unlock command is received, a YES path 866 may also be followed to acquire subsequent image data and block 854. Acquiring subsequent image data in Block 854 may include moving the ultrasound probe 400 and any appropriate manner. For example, the ultrasound probe 400 may move in a selected random or search pattern to acquire image data as selected according to a predetermined procedure. Further, the user 72 may input instructions regarding moving the ultrasound probe 400 to acquire further image data.

Regardless, the ultrasound probe 400 may be used to acquire image data of the subject 30. The ultrasound probe 400 may move based upon input from the user and/or based upon instructions according to the various processes, including the process 500 and/or the process 730 as discussed above.

Therefore, the ultrasound probe 400 may acquire image data of the subject, specific portions of the subject, portions of the instrument, or combinations thereof to be displayed for viewing by the user and/or for further analysis as discussed above.

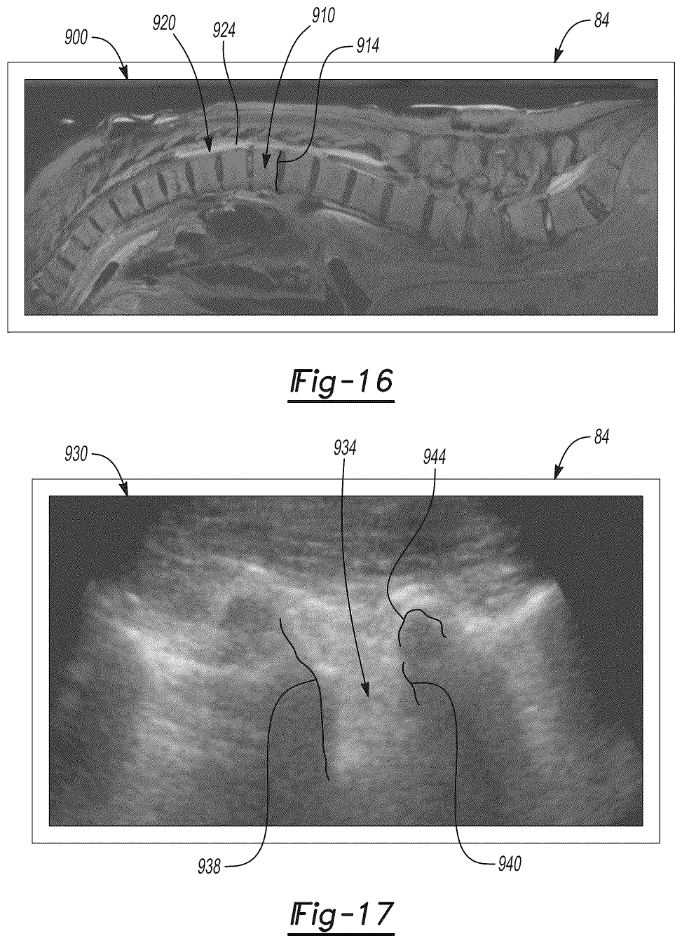

As discussed above data, such as image data, acquired of the subject 30 may be acquired with an ultrasound probe, according to various embodiments. For example, the ultrasound probe may be the imaging device 33 that is held and moved with the robotic system 20. In various embodiments, ultrasound arrays may include the ultrasound arrays 222 which, in various embodiments, may include scalar ultrasound arrays. Accordingly, one or many of the transducer assemblies (e.g., the ultrasound array 222) may be positioned relative to the subject either in a selected position and/or an immovable position to acquire data of the subject. The data acquired of the subject may be referred to as data or ultrasound data that may be used to generate images, such as sonograms of the subject 30. As also discussed above, the various ultrasound transducers or probes may be tracked with the navigation system 26 to allow for a determination of a pose of any acquired data with the ultrasound probe in the navigation space (e.g., the subject space or relative to the subject 30). Also, prior acquired image data or other image data may also be acquired of the subject 30, such as MR image data. Prior acquired image data may include any appropriate image data such as a MR image 900, as illustrated in FIG. 16. The MR image may be displayed on the display device 84, according to various embodiments. However, as discussed further herein, MR image 900 need not be displayed, but may be registered to the subject 30 and/or other data, such as data acquired with the ultrasound probe.

In various embodiments, the MR Image 900 may include data regarding the subject 30. For example, various structures, such as a first structure 910 may be included in the MRI image 900. The structure 910 may include a portion of a vertebral body of the subject 30. It is understood, however, that the structure 910 may be any appropriate structure such as any hard or rigid structure in any subject, such as a non-human subject. Further, various portions of the structure may be identified such as edges or boundaries 914.

The MR image 900 may also include non-rigid or hard regions 920 which may include various structures or boundaries 924 relative thereto. The non-rigid structures may be soft tissues of the subject 30, such as a spinal cord, musculature, vasculature, or other appropriate structures.

Nevertheless, the MR image 900 may be acquired with the subject. The MR image 900 may include any appropriate type of image data which may include three-dimensional data of the subject 30. The MR image 900 illustrated in FIG. 16 may be a two-dimensional rendering or slice of a three-dimensional image data acquisition and, therefore, is understood not to limit the type of MR image data acquired of the subject 30. The MR image 900 may include a selected expanse, such as all or most of the vertebrae within the subject 30. Accordingly, each of the original structures 910 may relate to one or more of the vertebrae and related bony structures such as a pelvis, ribs, or the like. Soft tissue portions 920 relative to the hard tissue portions may also be included in the MR image and provide for an illustration or representation of an expanse or portions of the subject 30. This allows the MR image 900 to include a region or volume that may provide a context to any particular portion included in the MR image, such as one or more of the vertebrae relative and inclusive of soft tissues and other portions surrounding the vertebrae.

One or more ultrasound probes, according to various embodiments, may generate an ultrasound image 930, as illustrated in FIG. 17. The ultrasound image 930 may be illustrated or displayed on the display device 84 for viewing by the user 72. However, as discussed further herein, the display of the ultrasound image is not required for various processes, such as analysis of the subject 30, registration to other data such as the MR image 900, or other appropriate purposes. Nevertheless, according to various embodiments, the ultrasound image 930 may include data that may be used for various purposes.

The ultrasound image 930 may be of one or more vertebra in the spine of the subject 30. The spine of the subject may be imaged with the ultrasound probe, according to various embodiments, to acquire data thereof. For example, as illustrated in the image 930, a vertebra 934 may be included in the data 930. The data 930 may be analyzed, such as with a selected trained machine learning algorithm, manual identification, other automatic algorithmic detection, or the like to identify or determine various features in the ultrasound image 930. As is generally understood by one skilled in the art, the ultrasound image 930 may include portions that are echogenic at the selected settings of the ultrasound transducers. Therefore, the image 930 may include portions of the vertebra 934 that may be identified, such as various features thereof. The features may include one or more boundaries of a vertebra, such as a first boundary 938 and a second boundary 940. The boundaries 938 and 940 may be used to identify various portions of the vertebra 934 and/or a specific vertebra. This identification may allow for registration to other image data, as discussed further herein. Additionally, other portions of the anatomy be identified, such as an articular region 944 that may be between two or more vertebra in the ultrasound image 930.

The ultrasound image 930 may be acquired of the subject 30 and various echogenic features of the subject 30 may be identified in the ultrasound image 930, such as various edges or contacts or interfaces between bony structures, such as the vertebrae 934 in other tissues. These features may be compared to other image data or images to assist in registering or correlating the ultrasound image 930 and the other images. As discussed above, the ultrasound probe may be tracked in the navigation system and therefore the pose of the identified portions in the ultrasound image may be known in the navigation space and allow for registration of the navigation space to other images once the ultrasound image 930 is registered to other images, such as the MR image 900.

To assist in registration of the MR image 900 with the ultrasound image 930, the MR image may be initially processed. The processing of the MR image, or any first image data, may include the generation of an intermediate or correlatable image data or image. The intermediate image data may be any appropriate image data and various embodiments are discussed herein. The intermediate image data may assist in the registration to a second image data and various embodiments are discussed herein.

In various embodiments, the MR image processing may include the generation of an echogenic feature map or space of the MR image. In various embodiments, for example, the identification of echogenic edges or surfaces in the MR image may be determined. For example, the edge 914 of the vertebrae 910 may be identified or processed in the MR image 900. Similarly or alternatively, edges or surfaces of soft tissue regions, such as the surface 924 may be identified or processed in the MR image 900.

In various embodiments, MR image 900 may also be used to generate or be processed to generate a mock computer tomography (mock CT) image or data space. The mock CT may be an exemplary data or feature space that is generated based upon the MR image 900 to allow for registration of the MR image 900 with the ultrasound image or data, such as the US image 930. The registration, as discussed further herein, may allow for a contextual understanding of various portions of the subject. The contextual image portions may allow identification of features that may not be efficiently identified in the ultrasound image. Further, the processed MR image, such as to the mock CT, may allow for registration of the MR image to the subject space due to or by the tracking of the ultrasound probe relative to the subject 30. Accordingly, as illustrated in FIG. 17, various features in an ultrasound may be identified, such as edges in an ultrasound sonogram. It is understood that an appropriate number, such as more than one, image data projections may also be combined into a three-dimensional ultrasound sonogram. Accordingly, for example, the plurality of arrays 222 associated with the subject 30, may be used to generate image data to allow for generation of a three-dimensional image based upon the ultrasound image data. Alternatively or additionally, moving the ultrasound probe, such as the ultrasound probe 400, to several positions relative to one another may allow for the generation of three-dimensional images based upon the acquired image data. Thus, the two-dimensional image as illustrated in FIG. 17 is merely an example as is the MR image 900 illustrated in FIG. 16 which may be a slice of a three-dimensional MR image. Nevertheless, a process may be applied to generate an image or image data that may be used to register or correlate the US image 930 and the MR image 900.

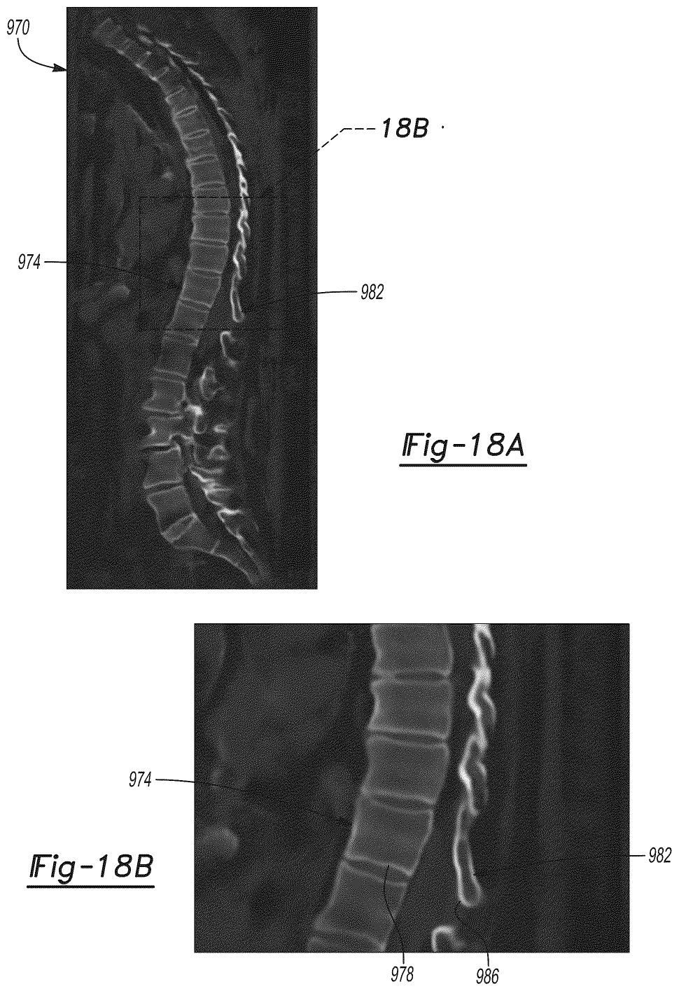

Turning reference to FIG. 18A, for example, a mock CT image or image portion 970 may be generated. The mock CT image 970 may include image portions that mimic or have features similar to a CT image. For example, hard tissue, such as a vertebral body 974 may be generated including a segmentation and identification of an edge 978 thereof, as illustrated in FIG. 18B. Additional features may also be identified such as a spinous process 982 and a related boundary or edge 986 thereof. The various portions, such as the vertebral body 974 and spinous process 982 may be identified or generated as a mock CT image portion based upon the MR image 900. In various embodiments, a trained algorithm, such as machine learning or artificial intelligence algorithm, may be trained on a plurality MR image data to generate the mock CT image or image data. In various embodiments, the mock CT image data may be displayed or not displayed. In various embodiments, the mock CT may be only generated for further analysis and comparison to an ultrasound image, as discussed further herein.

In addition or alternative to the mock CT, other image data or portions that are substantially similar to a CT image may be generated, such as including the identification of echogenic features in the MR image. For example, the identification of one or more echogenic boundaries, such as a boundary between soft tissue and hard tissue may be identified in the MR image 900. The echogenic boundary may be determined in a similar process as generation of the mock CT such as by an algorithm or trained algorithm. Further, the echogenic boundaries may allow for the generation of image data that does not require an entire generation of a mock CT image based upon the MR image data.

Alternatively or additionally, a manual identification or segmentation process may be used to determine various boundaries of the MR image. For example, a manual identification of echogenic or ultrasound imageable portions in the MR image may be manually identified by user. The manual identification may be saved relative to the MR image for later correlation to an ultrasound image. The ultrasound image data may be acquired at any appropriate time, such as during the procedure as discussed above. The ultrasound data may include the echogenic feature of the subject 30 that are identified in the MR image 900 to allow for the correlation thereto.

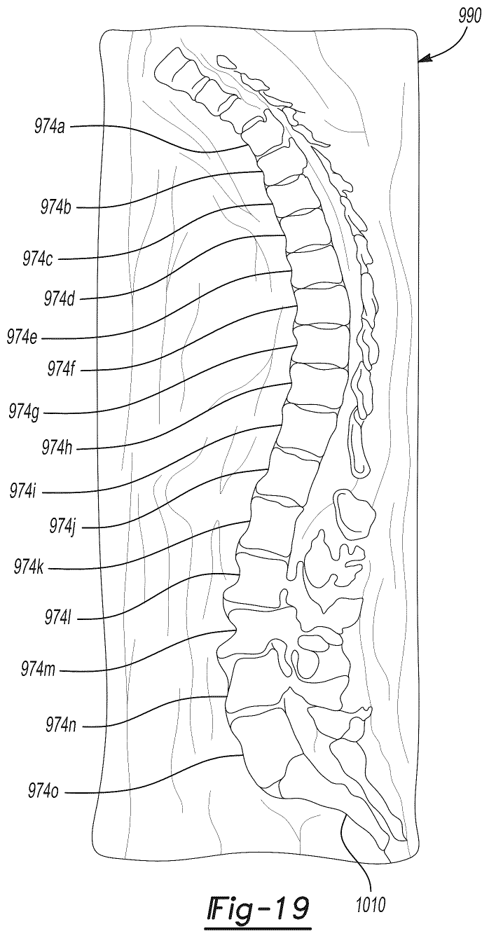

The generated image data, for example the mock CT image data, may then be segmented into various portions as segmented image data 990 as illustrated in FIG. 19. As discussed above, the image data may include a vertebra of the subject 30. The vertebra including the vertebral body 974, Several vertebrae, therefore, may be individually segmented into individual vertebra, such as vertebra 974a to 9740. Other anatomical features may also be identified or segmented in the mock CT image data or data, such as a sacrum 1010. It is understood, however, that any appropriate portion may be segmented or identified in the mock CT data. Therefore, also the various processes related to each of the vertebral bodies, or other portions, may be identified in the mock CT.

In various embodiments, for example, each of the vertebrae may be understood to be a unique or individual body, such as a rigid structure. More than one of the elements, however, may be understood to be a single or one rigid member, such as the for vertebral bodies 9740 and 974n. The two vertebral bodies 9740 and 974n may substantially be immovable relative to one another based upon various features, such as anatomy surrounding the vertebral bodies. Nevertheless, each may be segmented and identified as an individual rigid member, as illustrated in FIG. 19.

Further, each of the members may then be registered or correlated to other image data. As discussed above, the ultrasound image 930 may include various features or have identified features therein that may be registered to the segmented portions in the segmented image 990. Again the segmented image or image data 990 may be the segmented mock CT image data. Accordingly, the segmented image data 990 may be displayed and/or may not be displayed. Nevertheless, the segmented image 990 may be registered to one or more image data acquisitions of the ultrasound image data. Thus, the segmented image 990 may be registered to ultrasound image data (sonograms) and thereafter also to the navigation or patient space of the subject 30.

In various embodiments, for example, the ultrasound imaging system, such as the ultrasound probe 440 or the ultrasound arrays 222, may acquire ultrasound data or echogenic data from various portions of the subject 30, such as vertebrae in the subject. The ultrasound data may then be correlated or registered to the segmented portions in the segmented mock CT 990. The correlation may then allow for registration of the segmented mock CT image 990 to the ultrasound or echogenic data collected with the ultrasound probe.

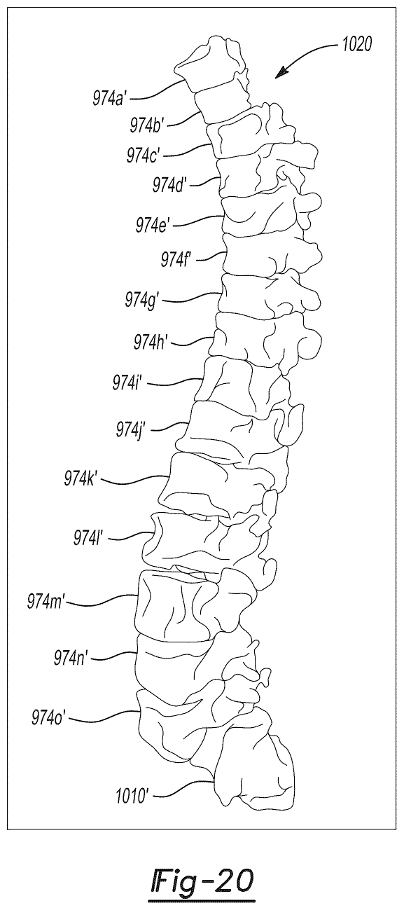

The segmented image 990, which may be registered to the tracked ultrasound data, may be used to generate the model 1020 as illustrated in FIG. 20. The model 1020 may be a selected model such as a two-dimensional model or a three-dimensional model. The model 1020 may be of the entire structure, such as of an entire or selected portion of the spine including multiple vertebral elements or several individual elements or members. For example, the model 1020 may include each individual element or member, such as each of the vertebra or vertebral bodies 974a′ to 9740′.

Each of the individual members may be individually registered and/or trackable based upon a local rigid registration. The registration may be based upon the registration of the data collected with the ultrasound, which may be two-dimensional data that is registered to the three-dimensional data, such as from the MR image 900. The production or generation of the mock CT image data or mock CT data may allow for the registration of the ultrasound data to the three-dimensional data of the MR image 900. Therefore, the generated model 1020 may be a three-dimensional model that is registered to the ultrasound data that is collected of the subject 30.

The ultrasound data collected of the subject 30 may be substantially real time image data. The registration of the real time image data allows for a real time registration of each of the individual members of the segmented image 990 in the model 1020. According to various embodiments, the individual or local registration may be based upon the processes such as those disclosed in U.S. Pat. No. 10,262,424 or U.S. Pat. No. 11,657,518, both incorporated herein by reference. This allows the model 1020 to illustrate a registered pose of each of the members that are identified and/or segmented in the mock CT image, such as from the segmented mock CT image 990. Thus, the pose of each of the individual members may be illustrated in the model 1020 such as each of the vertebral bodies 974a′ to 9740′ and/or the sacrum 1010′.

Due to the registration of the MR image 900 to the ultrasound image and the generation of the model 1020 based thereon, a registration and illustration of a current pose of the portions of the MR image 900 may be displayed. Further, various portions in addition to the registered portions, may be illustrated. The additional portions may include image data in addition to that of the individual vertebra 974. Additional image data may include non-segmented portions, such as soft tissues or other tissues. These may be illustrated relative to the model 1020 such as on the display 84. The model 1020, therefore, alone may be illustrated and/or illustrated relative to other image data, such as soft tissue image data such as based upon the MR image 900. The user 72 may view the registered model 1020, the MR image 900 registered to the subject based upon the segmented mock CT 990 and/or the registered model 1020, and related portion or features (such as anatomical portions including soft tissue) and in addition to other portions such as vertebrae.

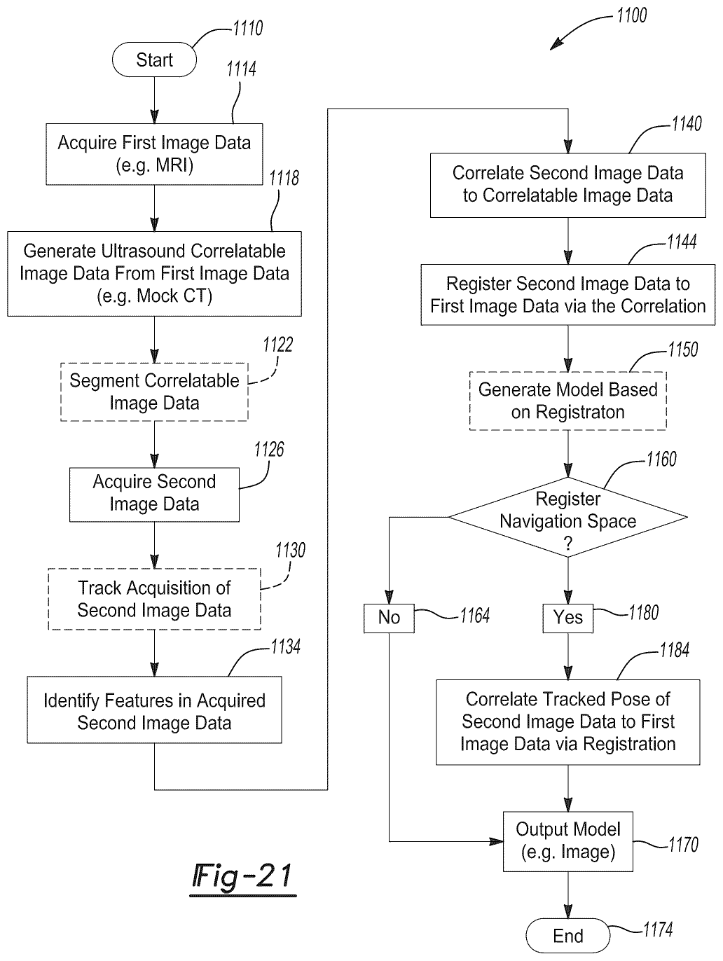

The various image data may be processed and registered images and/or generated model 1020 that may be registered may be generated, as discussed above, according to a process 1100 as illustrated in FIG. 21. The process 1100 may begin at START block 1110. In initiating the process of start block 1110, the user 72 or any appropriate individual may operate a processor module or system, including those discussed above such as the navigation system 26 including the processor module associated therewith. The process 1100, therefore, may be understood to be a process that is essentially automatically performed by execution of instructions of a processor module once initiated by a user. The process, however, may also include various input, such as from the user 72 or imaging systems or image data, as discussed further herein.

The process 1100 may acquire a first image data in block 1114. The first image data may include any appropriate image data, such as the MR image data 900. As discussed above, the first image data may be two-dimensional image data or three-dimensional image data. If three-dimensional image data, a slice or portion of the image data may be created therefrom that is 2-dimensional, if selected, for registration or correlation to other data. The image data acquired in block 1114 may be acquired at any appropriate time of the subject. In various embodiments, the image data may be a pre-acquired image data, such as acquired prior to a current or real time procedure. Alternatively or additionally, image data may be acquired in substantially real time of the subject. In various embodiments, for example, image data may be acquired with an intraoperative MR scanner to generate image data and images of the subject during a procedure, such as while instrument is positioned within the subject or relative to the subject 30. Thus, the process 1100 allows for the acquisition of first image in a selected manner in block 1114.

A generation of ultrasound correlatable image data from the first image data may then be generated in block 1118. The generation of the correlatable image data may include various processes that may generally allow for the generation, creation, or identification of data or portions in the first image data that may relate to echogenic data. For example, various edges or surfaces may be identified in the first image data that may be also acquired in an ultrasound image or data acquisition. In various embodiments, the correlatable image data may be a mock CT, such as the mock CT 970.

The mock CT may be generated on or based on the first image data with an appropriate process, such as with a trained machine learning system. The trained machine learning system may analyze or process the first image data to identify or generate an image or image data that mimics a computed tomography scan of the features included in the image data. In various embodiments, therefore, the mock CT image or image data may be generated based upon the first image data in block 1118.

The generation of the mock CT may be also performed substantially automatically, such as after the acquisition of the first image data in block 1114. Additionally or alternatively the generation of the mock CT image data may be generated at any appropriate time. Further, according to the discussion herein, the mock CT image data may be understood to relate to a specific type of data or image data but may also relate to a general process or data that may be correlated to an ultrasound image acquisition. Thus, the discussion herein, unless specifically indicated otherwise, may be understood to cover a general type of image data that may be generated block 1118. Thus mock CT image data may be understood to be an exemplary image data generation, unless specifically indicated otherwise.

The correlatable image data may then be optionally segmented in block 1122. The segmentation of the correlatable image data may allow for the segmentation of various features in the correlatable image data, such as specific portions of a vertebra or vertebrae as discussed above. The correlatable image data may be segmented to identify or determine various portions in the image data such as the vertebral body 974 or the spinous process 982, as noted above. Further various portions may be segmented to identify edges or surfaces thereof, as discussed above. The process may be based upon various generally known processes such as segmentation of computed tomography image data.

The process 1100 may then also allow for the acquisition of second image data in block 1126. The acquisition of the second image data in block 1126 may be image data that is generated or required with the ultrasound arrays, as discussed above. According to various embodiments, the ultrasound arrays, such as the arrays 222, may be used to generate ultrasound image data of the subject 30. Additionally or alternatively, the ultrasound probe, such as the probe 400, may be used to acquire the second image data. The second image data may be acquired of the subject 30 in an appropriate manner. The second image data may include ultrasound image data, as discussed above.

During the acquisition of the second image data, the imaging system may be optionally tracked in block 1130. Tracking the acquisition of the second image data may include tracking the ultrasound probe 400, tracking the arrays 222, or any appropriate tracking process. As discussed above, the various ultrasound systems may be tracked in a navigation space. The navigation space may include, be registered to, or be defined relative to the subject 30. Therefore, the ultrasound arrays may generate image data that is at a known or registered pose relative to the subject 30. The imaging system and the acquired image data may be registered to the subject 30, such as for navigation and/or registration of other coordinate systems, such as of the first image data as discussed further herein according the process 1100.

Features may be identified in the second image data in block 1134. Features in the second image data may include various features, such as specific portions including a vertebra and/or edges or features, such as the edge 938 or articulation surface 944 as illustrated in FIG. 17 and discussed above. The various identified features may be used for correlation to the correlatable image data such that the second image data may be correlated to the correlatable image data in block 1140.

The correlation of the second image dated to correlatable image data in block 1140 may be based upon the generated correlatable image data and the identified features from block 1134. The correlation may include the identification or selection of features or portions that exist in both of the second image data and the correlatable image data. For example, the articulation surface 944 that is identified in the ultrasound image, as illustrated in FIG. 17, may also be identified in the correlatable image data, such as the mock CT as illustrated in FIGS. 18A and 18B. Thus, the second image data and the correlatable image data may be correlated in block 1140 by allowing for a match or correlation of the various features in both of the image data sets. In other words, the correlation in block 1140 may be correlating the intermediate image data to the second image data by relating features identified in both the second image data and the intermediate image data.

The second image data may then be registered to the first image data in block 1144. The registration of the first image data to the second image data may be based upon the correlation of the second image data features to the correlatable image data in block1140. As the correlatable image data is based or generated from the first image data the pose or coordinates of features in the first image data may be identified in the first image data based upon the correlatable image data. Thus, once the correlation of the second image data to the correlatable image data is made in block 1140 registration may be made between the second image data and the first image data. This may allow for the registration of the first and second image data for various purposes, as discussed herein. According to various embodiments, for example, an optional generation of a model may be based upon the registration in block 1150. The model may include the model, as discussed above, in the model 1020.

The model may allow for a display of the model 1020 on the display device 84, as discussed above. In various embodiments generation of the model may allow for clarity of the display, ease of manipulation of the display, or other appropriate purposes. Nevertheless the model may be generated in block 1150. The model may be a generated image based on the registration, but entirely generated for the display. In various embodiments, however, registration of the second image data to the first image data may allow for updating of first image data, such as a segmented portion thereof, to illustrate a current or real time pose of portions in the first image data to match the current pose of the subject 30.

A determination may be made of whether to register the first image data to navigation space in block 1160. If a determination is made that registration will not occur a NO path 1164 may be followed to an output in block 1170. The output in block 1170 may be of various types such as outputting to memory for later recall, outputting the generated model from block 1150 if generated or selected, or output of a registered image based upon the registration from block 1144. Thus, the user 72 may save the output from block 1170 for various purposes and/or allow for display of the output. The process 1100 may then end in END block 1174. The ending of the process in block 1174, however, may allow for various other actions to occur, such as illustration of image data of the subject, planning of a procedure relative to the subject, or other various purposes, including those also discussed herein.

According to various embodiments, registration may be determined to occur in block 1160 and then a YES path 1180 may be followed. The YES path 1180 may allow for a correlation of the tracked pose of the second image data to the first image data via the registration in block 1184. Thus, the output may then follow the correlation in block 1170 and include a registered or updated pose of the first image data based upon the tracked pose in the second image data. Therefore, the output may include a display of various portions, such as the model, the image, or the like to illustrate portions in a current or real time pose based upon the tracked acquisition of the second image data in block 1130 and the correlation thereof in block 1184. Thus, for example, the model 1020 as illustrated in FIG. 20 may be illustrating a current or real time pose of the various displayed elements, such as the segmented vertebrae. Further, as discussed above, each of the individual vertebrae may be individually tracked, based on the second image data, and displayed based upon a local registration thereof. This may allow the user 72 to understand a real time pose of the various features that are imaged in the first image data based upon the track portion and the second image data. Additionally, as discussed above, various features may be illustrated relative to the model portions 1020 and/or the first image data may be displayed with the registered features thereof to allow the user 72 to understand a context of the tracked or registered portions. Further the first image data may include greater context or volume than the second image data, such as may be included in MR image data as compared to ultrasound image data. Thus the registration or correlation in block 1184 may allow for the real time illustration or display of the first image data, such as the MR image 900, based upon or updated with real time image data acquired with the ultrasound systems.

Therefore, the process 1100 may allow for the registration of a first and second image data. Further the registration of the first and second image data may allow for the generation and display of real time poses of features in the first image data based upon the second image data. This may allow the user 72 to better understand a current or real time pose of various portions, such as based upon the acquisition with the second image data of image data of the subject while the imaging system is being tracked.

Also, the END block 1174 may be an end of one iteration of the process 1100. Thus, the user 72 or other appropriate user may select perform the process 1100 again. In various embodiments, the process 1100 may also be performed automatically based on the collection of second image data. Thus, the real time pose and registration or updated display may be made by operation of the process 1100 continuously or at a selected interval. Thus, the process 1100 may be understood to be a single iteration and may be performed at a selected rate and/or a selected number of times.

According to various embodiments, the ultrasound probe may emit or transmit ultrasound waves in a selected pattern or plane. The plane may be a shape as is understood by one skilled in the art. The plane is generally able to acquire data in a field of view to generate images, also referred to as sonograms when images are generated based on ultrasound data.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method of registering a first image data to a second image data via an intermediate image data, the method comprising:
acquiring a first image data;
acquiring a second image data;
generating the intermediate image data that is correlatable to the second image data from the first image data;
correlating the intermediate image data to the second image data; and
registering the first image data to the second image data.

2. The method of claim 1, wherein acquiring the first image data includes acquiring magnetic resonance image data.

3. The method of claim 2, wherein generating the intermediate image data that is correlatable to the second image data from the first image data includes generating a mock computed tomography image data based on the first image data.

4. The method of claim 2, wherein generating the intermediate image data that is correlatable to the second image data from the first image data includes identifying echogenic features in the first image data.

5. The method of claim 1, wherein generating the intermediate image data that is correlatable to the second image data from the first image data is automatically generated by executing instructions of an algorithm with a processor module.

6. The method of claim 1, wherein generating the intermediate image data that is correlatable to the second image data from the first image data is automatically generated by processing the first image data with a trained model.

7. The method of claim 1, wherein correlating the intermediate image data to the second image data includes relating features identified in both the second image data and the intermediate image data.

8. The method of claim 7, wherein registering the first image data to the second image data includes defining a translation between a first image coordinate system and a second image coordinate system based on the correlation of the intermediate image data to the second image data.

9. The method of claim 8, further comprising:
registering the first image coordinate system to a subject coordinate system.

10. The method of claim 9, further comprising:
tracking an imaging system in the subject coordinate system; and
wherein the acquired second image data is acquired with the imaging system while being tracked.

11. The method of claim 1, further comprising:
tracking an imaging system in a subject coordinate system while the imaging system is acquiring the second image data while being tracked;
registering the first image data to the subject coordinate system based on the registration of the first image data to the second image data.

12. A system to register a first image data to a second image data via an intermediate image data, comprising:
a processor module configured to execute instructions to:
acquire a first image data;
acquire a second image data;
generate the intermediate image data that is correlatable to the second image data from the first image data;
correlate the intermediate image data to the second image data; and
register the first image data to the second image data.

13. The system of claim 12, wherein the first image data is magnetic resonance image data;
wherein the processor module is configured to execute further instructions to generate a mock computed tomography image data based on the first image data as the intermediate image data that is correlatable to the second image data from the first image data includes.

14. The system of claim 12, wherein the first image data is magnetic resonance image data;
wherein the processor module is configured to execute further instructions to identify echogenic features in the first image data to generate the intermediate image data that is correlatable to the second image data from the first image data includes.

15. The system of claim 12, further comprising:
a memory system having stored thereon a trained model;
wherein the intermediate image data that is correlatable to the second image data from the first image data is automatically generated by processing the first image data with the trained model.

16. The system of claim 12, wherein the processor module is configured to execute further instructions to:
relate features in the second image data to features in the intermediate image data to correlate the intermediate image data to the second image data includes;
define a translation between a first image coordinate system and a second image coordinate system based on the correlation of the intermediate image data to the second image data to register the first image data to the second image data includes.

17. The system of claim 16, wherein the processor module is configured to execute further instructions to:
register the first image coordinate system to a subject coordinate system.

18. The system of claim 17, further comprising:
a tracking system to track an imaging system in the subject coordinate system; and
wherein the acquired second image data is acquired with the imaging system while being tracked.

19. The system of claim 12, further comprising:
an imaging system to acquire the second image data; and
a tracking system to track the imaging system in a subject coordinate system while the imaging system is acquiring the second image data;
wherein the processor module is configured to execute further instructions to register the first image data to the subject coordinate system based on the registration of the first image data to the second image data.

20. The system of claim 12, further comprising:
a first imaging system to acquire the first image data;
a second imaging system to acquire the second image data;
a navigation system to track at least the second imaging system in a subject coordinate space while acquiring the second image data to allow a registration of the first image data to the subject coordinate system.
